(19) United States
(12) Patent Application Publication
(10) Pub. No.: US 2013/0053435 A1
(43) Pub. Date: Feb. 28, 2013

(54) Methods of Treating Alcohol Intoxication, Alcohol Use Disorders and Alcohol Abuse Which Comprises the Administration of Dihydromyricetin

(71) Applicants: Jing Liang, Los Angeles, CA (US); Richard W. Olsen, Los Angeles, CA (US); Igor Spigelman, Los Angeles, CA (US)

(72) Inventors: Jing Liang, Los Angeles, CA (US); Richard W. Olsen, Los Angeles, CA (US); Igor Spigelman, Los Angeles, CA (US)

(21) Appl. No.: 13/520,727

(22) PCT Filed: Aug. 23, 2011
(86) PCT No.: PCT/US11/48749

§ 371 Date: Jul. 5, 2012

(51) Int. Cl. A61K 31/352; A61P 39/02; C12N 5/079; A61P 25/32

(52) U.S. Cl. 514/456; 435/375

(57) ABSTRACT

Disclosed herein are methods for treating alcohol intoxication, alcohol use disorders and alcohol abuse which comprise the administration of dihydromyricetin. As disclosed herein, dihydromyricetin potentiates the activity of GABA_{A}Rs having the alpha4-beta-delta subunit which is associated with the effects of ethanol, antagonizes the actions of ethanol on the GABA_{A}Rs, acts on the benzodiazepine sites of GABA_{A}Rs, and inhibits, reduces and/or reverses some or all of the GABA_{A}R plasticity which is caused by exposure to ethanol.

### CROSS-REFERENCE TO RELATED APPLICATIONS

The present invention claims the benefit U.S. Patent Application Ser. No. 61/376,528, filed 24 Aug. 2010, which is herein incorporated by reference in its entirety.

### ACKNOWLEDGEMENT OF GOVERNMENT SUPPORT

This invention was made with Government support under Grant Nos. AA007680, AA016100 and AA0017991, awarded by the National Institutes of Health. The Government has certain rights in this invention.

### BACKGROUND OF THE INVENTION

1. Field of the Invention

The present invention generally relates to methods of using dihydromyricetin to modulate ethanol induced plasticity of γ-aminobutyric acid (A) receptors. The present invention also relates to methods of using dihydromyricetin to treat ethanol intoxication, alcohol use disorders and alcohol abuse.

2. Description of the Related Art

Alcohol dependence ranks third on the list of preventable causes of morbidity and mortality in the United States. There are more than 20,000 alcohol-induced deaths every year in the United States excluding accidents and homicides. In 2008, 11,773 people were killed in alcohol-impaired driving crashes, accounting for nearly one-third of all traffic-related deaths in the United States. According to the Centers for Disease Control and Prevention, the annual cost of alcohol-related crashes totals more than $51 billion.

Alcohol (ethanol, EtOH) interaction with γ-aminobutyric acid (A) receptors (GABA_{A}Rs) plays a role in alcohol withdrawal syndrome (AWS). See Becker H C (1998) Alcohol Health Res World 22(1):25-33; Boehm S L, 2nd, et al. (2004) Biochem Pharmacol 68(8):1581-1602; Koob G F (2004) Biochem Pharmacol 68:1515-1525; Anacker A M and Ryabinin A E (2010) Int J Environ Res Public Health 7(2):473-493; and Dopico A M and Lovinger D M (2009) Pharmacol Rev 61(1):98-114.

GABA_{A}Rs on synapses are formed of αβγ subunits which have low sensitivity to ethanol; while GABA_{A}Rs containing α4βδ subunits are highly sensitive to low ethanol concentrations. See Liang J, et al. (2008) Alcohol Clin Exp Res 32(1):19-26; Santhakumar V, et al. (2007) Alcohol 41(3):211-221; and Jia F et al. (2005) J Neurophysiol 94(6):4491-4501. GABA_{A}Rs are known to undergo allosteric modulation by ethanol, general anesthetics, benzodiazepines and neurosteroids. See Olsen R W and Homanics G E (2000) GABA IN THE NERVOUS SYSTEM: THE VIEW AT FIFTY YEARS (Martin D L and Olsen R W eds) pp 81-96, Lippincott Williams & Wilkins, Philadelphia; and Wallner M, et al. (2003) PNAS USA 100(25):15218-15223. The studies indicate that the underlying mechanism of AWS is GABA_{A}Rs plasticity induced by excessive abuse of ethanol, which is associated with generally decreased GABA_{A}R activation and differentially altered subunit expression. See Olsen R W, et al. (2005) Neurochem Res 30:1579-1588; Liang J, et al. (2006) J Neurosci 26:1749-1758; and Liang J, et al. (2007) J Neurosci 27:12367-12377. Extrasynaptic α4βδ subunit containing GABA_{A}Rs internalize soon after ethanol intoxication in vitro and in vivo. See Shen Y, et al. (2010) Mol Pharmacol 79(3):432-442; and Liang J, et al. (2007). Extrasynaptic α4βδ subunit containing GABA_{A}Rs exhibit significant linear relationship with behavioral loss of righting reflex (LORR) induced by ethanol intoxication and other sedative-hypnotic-anesthetic drugs. See Liang J, et al. (2009) J Neurophysiol 102:224-233. In other words, extrasynaptic α4βδ containing GABA_{A}R property changes underlie alcohol-induced behavioral changes. Thus, GABA_{A}Rs have been indicated as a possible neuropharmacological target in the treatment of alcohol dependence. See Olsen R W and Sieghart W (2009) Neuropharmacology 56:141-148. Unfortunately, there are no known methods or compositions which inhibit and/or reverse GABA_{A}R plasticity caused by chronic exposure to ethanol.

Benzodiazepines (e.g. diazepam) are classical medications for reducing symptoms of AWS. However, benzodiazepines are inactive at the alcohol-sensitive, and insensitive α4βδ subunit-containing GABA_{A}Rs. In addition, benzodiazepines produce cross-tolerance to ethanol. Moreover, as a major side effect, frequent use of benzodiazepines can lead to dependence. In fact, the combination of benzodiazepines and alcohol cause even greater substance addiction problems which are more difficult to overcome as compared to alcohol dependence itself.

Besides benzodiazepines, only three medications, i.e. naltrexone, acamprosate, and disulfiram, are currently approved by the U.S. FDA for treating alcohol dependence. Naltrexone blocks opioid receptors and it may also impair thinking and reaction-time, and produce anxiety and other unhappy feelings. Acamprosate causes side effects including headache, diarrhea, flatulence and nausea and two large U.S. clinical trials failed to confirm its efficacy. Disulfiram is directed towards blocking the metabolism of alcohol, thereby causing a negative reaction to alcohol intake, and its side effects include flushing, accelerated heart rate, shortness of breath, nausea, vomiting, headaches, visual disturbances, mental confusion, and circulatory collapse. Disulfiram may also cause peripheral neuropathy.

Thus, a need exists for compositions and methods which treat, inhibit, reduce and/or reverse some or all GABA_{A}R plasticity caused by exposure to ethanol.

### SUMMARY OF THE INVENTION

In some embodiments, the present invention provides methods of treating, inhibiting, reducing and/or reversing GABA_{A}R plasticity caused by exposure to ethanol, which comprises administering dihydromyricetin to a GABA_{A} receptor that will be, is, and/or has been exposed to ethanol. In some embodiments, the present invention provides methods of potentiating the activity of GABA_{A} receptors, which comprises administering dihydromyricetin to the GABA_{A} receptor. In some embodiments, the present invention provides methods of antagonizing the activity of ethanol on GABA_{A} receptors, which comprises administering dihydromyricetin to the brain tissue acting on central nervous system GABA_{A} receptors before, during, and/or after exposure to the ethanol.

In some embodiments, the present invention provides methods of treating, inhibiting, and/or reducing ethanol intoxication, at least one symptom of alcohol withdrawal syndrome, alcohol use disorders and/or alcohol abuse in a subject, which comprises treating, inhibiting, reducing and/or reversing GABA_{A}R plasticity of the GABA_{A} receptors, potentiating the activity of the GABA_{A} receptors, and/or antagonizing the activity of ethanol on the GABA_{A} receptors as disclosed herein. In some embodiments, the subject is mammalian, preferably human. In some embodiments, the symptom of alcohol withdrawal syndrome is selected from the group consisting of tolerance to ethanol, increased basal anxiety, and hyperexcitability. In some embodiments, the treatment reduces or inhibits a decrease in alertness, in the subject, which is caused by the exposure to ethanol. In some embodiments, the alcohol abuse is high alcohol consumption that is induced by alcohol exposure.

In the embodiments disclosed herein, dihydromyricetin may be administered before, during and/or after the exposure to ethanol. In some embodiments, dihydromyricetin is administered during a period ranging from about 30 minutes to directly before exposure to ethanol. In some embodiments, dihydromyricetin is administered during a period ranging from directly after exposure to ethanol to about 30 minutes after exposure to ethanol. In some embodiments, dihydromyricetin may be administered in the form of a foodstuff, such as a beverage, which may or may not contain ethanol. In some embodiments, dihydromyricetin may be administered in the form of a pharmaceutical formulation. In some embodiments, dihydromyricetin is co-administered with ethanol. In the embodiments disclosed herein, dihydromyricetin may be administered in an effective amount. In some embodiments, dihydromyricetin is administered in a therapeutically effective amount. In some embodiments, dihydromyricetin is administered in a unit-dosage form. In some environments, the amount of dihydromyricetin in a unit-dosage form for a human is about 50-70 mg.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description serve to explain the principles of the invention.

### DESCRIPTION OF THE DRAWINGS

This invention is further understood by reference to the drawings wherein:

FIGS. 1A-1F are graphs showing that DHM blocks acute EtOH intoxication and prevents EtOH withdrawal symptoms. Reduction in EtOH (3 g/kg, i.p.) induced LORR duration by pre- (FIG. 1A), post- (FIG. 1B) and combined- (FIG. 1C) treatment with DHM (1 mg/kg, i.p., n=5-7 rats/group). Vehicle rats received saline (20 ml/kg, i.p.). The results show that DHM alone does not induce LORR (FIG. 1C), yet even when injected 30 min post-EtOH (dashed line), DHM significantly reduces LORR duration. FIG. 1D is a graph showing the results of a separate experiment. As shown in FIG. 1D, concurrent i.p. injection of EtOH (3 g/kg) and DHM (1 mg/kg, i.p.) abolishes withdrawal (48 hr)-induced tolerance to EtOH (3 g/kg, i.p.)-induced LORR (n=7/group). Co-administration of DHM+EtOH prevents withdrawal (24 hr)-induced increases in PTZ-induced seizure duration (FIG. 1E) and incidence (FIG. 1F) from single-dose EtOH intoxication (same animals as in FIG. 1B). *, p<0.01 vs. vehicle-treated, one-way ANOVA.

FIGS. 2A-2B are graphs showing that DHM prevents single-dose EtOH exposure/withdrawal-induced GABA_{A}R plasticity. Rats were divided into 4 groups and gavaged with either vehicle, EtOH (5 g/kg, E), EtOH combined with DHM (1 mg/Kg, E+D) or DHM (D). After 48 hr withdrawal, patch-clamp recordings were performed on DGCs in hippocampal slices. The I_{tonic} changes are shown in FIG. 2A, the mIPSCs changes are shown in FIG. 2B (% control). n=4-7 rats/group. *, p<0.05 vs. 0; †, p<0.05 vs. vehicle-treated, two-way RM ANOVA.

FIG. 3 are graphs showing that DHM enhances GABA_{A}R-mediated currents, and antagonizes their potentiation by acute EtOH in DGCs from naïve rats. Panel-a is a continuous current trace showing the effect of DHM on I_{tonic} magnitude and mIPSC charge transfer (mIPSC area). Total charge transfer is slightly enhanced by DHM (1.0 μM). DHM concentration-dependent potentiation of I_{tonic} (panel a-1) and mIPSCs (panel a-2). n=6 neurons/group. *, p<0.05 vs. pre-drug, one-way ANOVA. Panel B is a sample trace recording from a DGC during application of EtOH (60 mM) followed by EtOH co-application with DHM (0.3 and 1 μM). Panel b-1 shows that I_{tonic} magnitudes are significantly enhanced by EtOH; this enhancement is concentration-dependently reduced by DHM co-application. n=6 neurons/group. Panel b-2 shows that mIPSC total charge transfer is similarly affected by EtOH−DHM, but due to the low sensitivity of mIPSCs to both EtOH and DHM the effects are not significant. n=5-7 neurons/group. Panel c shows a sample trace recording from a DGC during application of DHM (0.3 μM) followed by co-application of DHM with EtOH (10 and 60 mM). Panel c-1 shows that EtOH does not affect I_{tonic} potentiation by DHM. Panel c-2 shows that mIPSCs total charge transfer is similarly affected by DHM-EtOH but the effects are not significant. n=5-7 neurons/group. *, p<0.01, post-DHM vs. pre-drug, one-way ANOVA.

FIGS. 4A-4B show that co-administration of DHM+EtOH prevents EtOH intoxication-induced functional GABA_{A}R plasticity in DIV14 primary cultured hippocampal neurons. FIG. 4A is a summary of I_{tonic} magnitude and FIG. 4B shows changes of mIPSC charge transfer (% pre-drug) in response to acute EtOH (60 mM) from vehicle-, EtOH−, EtOH+DHM- and DHM-treated neurons. n=8-9 neurons/group. *, p<0.05, vs. pre-EtOH; †, p<0.05, drug-treated vs. vehicle-treated, two-way RM ANOVA.

FIGS. 5A-5D show that DHM potentiates GABA_{A}R function in both control and EtOH exposure/withdrawal neurons. DHM concentration-dependently enhanced GABA_{A}R-mediated I_{tonic} (FIG. 5A) and mIPSCs (FIG. 5B) in DIV14 neurons. The response is modestly decreased after EtOH exposure (closed circles) compared to control (open circles). There is a slight right shift in I_{tonic} magnitude but not in mIPSC total charge transfer after EtOH exposure/withdrawal (n=5-9 neurons/group). FIG. 5C shows sample traces of evoked-GABA_{A}R-mediated currents. FIG. 5D shows the effect of DHM on the GABA concentration-response curve. Amplitudes are normalized to the peak current activated by 300 μM GABA in the absence of DHM. Each data point is the average amplitude from 5 to 9 neurons. DHM was co-applied with GABA.

FIGS. 6A-6C are graphs showing that DHM counteracts EtOH intoxication and the effects of DHM are antagonized by flumazenil. FIG. 6A shows that EtOH (E, 3 g/kg, i.p. injection) induced Loss-of Righting Reflex (LORR), while concurrent injection of DHM (1 mg/kg, i.p.) with EtOH (E+D1) greatly reduced the duration of LORR. DHM (D1), as a saline injection, did not induce LORR (n=8-20 rats/group). FIG. 6B shows that DHM application 30 min prior to EtOH injection counteracted EtOH-induced LORR; While 30 min after EtOH-induced LORR (indicated as black lines), DHM injection reduced the residue of LORR (n=5-10 rats/group). FIG. 6C shows that co-injection of EtOH and DHM (3 mg/kg, E+D3) greatly reduced the EtOH-induced LORR. Concurrent injection of flumazenil (10 mg/kg, F10), EtOH and DHM (E+D3+F10), reversed DHM effects. When the dose of DHM was increased to 10 mg/kg (E+D10+F10), flumazenil partially reversed the effects of DHM. When the dose of flumazenil was increased to 30 mg/kg (E+D3+F30), stronger antagonism of DHM was observed. Co-injection of flumazenil with EtOH (E+F10) did not alter LORR duration (n=5-6 rats/group, †, p<0.05 vs. saline group, *, p<0.05 vs. EtOH group).

FIG. 7 shows the effects of high dosages of DHM and flumazenil on LORR in rats. 100 or 300 mg/kg DHM (i.p. injection) induced very short LORR duration. i.p. injection of flumazenil at 30 and 200 mg/kg did not induce LORR.

FIG. 8 shows results of a plasma [EtOH] assay during EtOH-induced LORR. X axis shows blood sampling time points after i.p. injection of EtOH (3 g/kg) or co-application of DHM (1 and 10 mg/kg) with EtOH (E+D) (n=3-4 rats/group, *, p<0.05 vs. EtOH group, two-way RM ANOVA).

FIG. 9 shows that DHM antagonizes EtOH-induced GABA_{A}R potentiation and the effect is blocked by flumazenil. Panel A shows whole-cell voltage-clamp (−70 mV) recording from rat hippocampal DGCs (left) and superimposed averaged mIPSCs (right). The gray dashed lines represent the mean currents after complete blockade of all GABA_{A}R-currents by picrotoxin (PTX, a GABA_{A}R antagonist, 100 μM) as a baseline to calculate the magnitude of GABA_{A}R-mediated I_{tonic}. Bath application of EtOH (60 Mm, E) increased I_{tonic} and mIPSCs. DHM (0.3 and 1.0 μM) antagonized these EtOH effects. Panel B summarizes the I_{tonic} area in response to EtOH and DHM. Panel C shows the mIPSC area in response to EtOH and DHM. Panel D is a sample trace recorded from DGCs (left) and superimposed averaged mIPSCs (right). DHM (3 μM) antagonism of acute EtOH-induced GABA_{A}R potentiation was reversed by 10 μM flumazenil. Panel E is a summary of the I_{tonic} area in response to EtOH, DHM and flumazenil. Panel F is a summary of the mIPSC area in response to EtOH, DHM and flumazenil. n=4-6/group. *, p<0.05 vs. drug 0; †, p<0.05 vs. EtOH, two-way RM ANOVA.

FIG. 10 shows that DHM is a positive modulator of GABA_{A}Rs at benzodiazepine sites. Panel A shows the whole-cell voltage-clamp (−70 mV) recording from rats' hippocampal DGCs (left) and superimposed averaged mIPSCs (right). Panel B is a summary of I_{tonic} potentiated by DHM from 0.1 to 30 μM (n=4-5. *, p<0.05 vs. drug 0, one-way RM ANOVA). Panel C is a summary of mIPSC area potentiated by DHM from 0.1 to 30 μM (n=4-5. *, p<0.05 vs. drug 0, one-way RM ANOVA). Panel D shows the whole-cell voltage-clamp (−70 mV) recording from a cultured hippocampal neuron at DIV14 (DIV: days in vitro). DHM (1 μM, D1) enhanced GABA_{A}R-mediated I_{tonic} and mIPSCs were reversed by flumazenil (F, 10 and 100 μM). All GABA_{A}R-currents are blocked by bicuculline (GABA_{A}R antagonist, Bic, 10 μM, gray dashed line). Summary (% of pre-drug (0)) of DHM (1 μM, D1) enhancement of I_{tonic} (panel E) and mIPSCs (panel F), while flumazenil inhibited them concentration-dependently (n=7, *p<0.05 vs. drug 0, one-way RM ANOVA). Panel G shows that DHM inhibited [³H]flunitrazepam (flu) binding in rat cortex membrane homogenates. Increasing the final concentrations of DHM (0.03-100 μM) results in displacement of [³H]flunitrazepam (final concentration of 1 nM) at cortical binding sites. Results are graphed by GraphPad Prism 4.0 and presented as average of two experiments with each point done in triplicate (n=2).

FIGS. 11A-11D show that DHM potentiates GABA_{A}R-mediated inhibition in a concentration-dependent manner in DIV14 primary cultured hippocampal neurons from rats. DHM potentiates GABA_{A}R-mediated inhibition in a concentration-dependent manner. Cultured neurons at DIV14 were whole-cell voltage-clamped at −70 mV. Dose-response curves of DHM on I_{tonic} (FIG. 11A) and mIPSCs (FIG. 11B) (n=9-10 neurons/group). FIG. 11C shows sample traces from a cultured hippocampal neuron, showing DHM (1 μM) enhanced GABA_{A}R-currents evoked by focal puffs of 10 and 300 μM GABA. FIG. 11D shows the concentration-response curve of GABA_{A}R-currents induced by focal puffs of GABA was left-shifted by DHM (0.3 and 1 μM, n=5-9 neurons/group, *, p<0.05 vs. DHM 0, one-way ANOVA).

FIG. 12A-12E show that DHM prevents EtOH withdrawal symptoms and antagonizes EtOH exposure/withdrawal-induced alteration in GABA_{A}R α4 subunit expression in rat hippocampus. 4 groups of rats were injected (i.p.) with single-dose vehicle, EtOH (3 g/kg, E), EtOH plus DHM (1 mg/kg, E+D), or DHM alone (FIG. 12 D). After 48 hr withdrawal: FIG. 12A anxiety was measured by elevated plus maze (EPM). E-group spent shorter time in the open arms and longer time in the closed arms. E+D-group spent similar time in both arms as vehicle-group; FIG. 12B shows tolerance measured by LORR. E-group showed significant shorter duration of acute EtOH-induced LORR. E+D-group showed no different in LORR compared with vehicle-group; FIG. 12C shows that E-group increased PTZ-induced seizure duration. E+D-group showed similar PTZ-induced seizures as vehicle-group. D-group showed no difference compared with vehicle-group in all three assays (n=5-13 rats/group). FIG. 12D shows Western blots of hippocampal tissue GABA_{A}R α4 subunit after 48 hr withdrawal from rats gavaged with vehicle, EtOH, E+D or DHM. β-actin is shown as loading control. FIG. 12E shows the quantification of total α4 subunit protein from the experiments of FIG. 12D. EtOH-withdrawal induced an increase in α4 GABA_{A}R subunit, while E+D-treatment prevented this increase. DHM did not produce changes in α4 GABA_{A}R subunit protein (n=3 rats/group, *, p<0.05 vs. vehicle-treated, one-way ANOVA). [symbol] =vehicle, [symbol] =EtOH, [symbol] =EtOH+DHM, [symbol] =DHM.

FIG. 13 shows that DHM inhibits EtOH exposure/withdrawal-induced GABA_{A}R functional plasticity in hippocampal DGCs in rats. Rats were divided into 4 groups and gavaged with either vehicle, EtOH (5 g/kg, E), EtOH combined with DHM (1 mg/Kg, E+D) or DHM (panel D). After 48 hr withdrawal, patch-clamp recordings were performed on DGCs in hippocampal slices. Panel A shows acute EtOH (60 mM) enhanced I_{tonic} and mIPSCs in vehicle-treated rats. Panel B shows that in the EtOH/withdrawal group, EtOH did not increase I_{tonic} while it greatly enhanced mIPSC area. Panel C shows that in the E+D group, EtOH increased I_{tonic} and mIPSCs similar to those of the vehicle group. Panel D shows the responses of I_{tonic} and mIPSCs to EtOH from the DHM group were similar to those of the vehicle group. Panel E and F show that Zolpidem (ZP, a benzodiazepine agonist, 0.3 μM) potentiated I_{tonic} and mIPSCs in the DHM group as in vehicle group; while it did not affect GABA_{A}R-currents in the EtOH group. Panel G shows a summary of EtOH effects on I_{tonic} in the 4 groups. Panel H shows a summary of EtOH effects on mIPSCs in the 4 groups. Panel I shows a summary of zolpidem effects on I_{tonic} and panel J shows a summary of the mIPSC area in the 4 groups (n=4-7 rats/group, *, p<0.05 vs. drug 0; †, p<0.05 vs. vehicle group, two-way RM ANOVA).

FIGS. 14A-14D show that DHM potentiates GABA_{A}R-mediated inhibition in EtOH pre-exposed cultured hippocampal neurons; Co-administration of DHM with EtOH prevents EtOH-induced GABA_{A}R plasticity in vitro. In culture hippocampal neurons (DIV13-14) 24 hr after EtOH-exposure (60 mM, 30 min), DHM can still enhanced both GABA_{A}R-mediated I_{tonic} (FIG. 14A) and mIPSC area (FIG. 14B) concentration-dependently without tolerance compared with FIGS. 11A and 11B (n=8-9 neurons/group, *, p<0.05 vs. drug 0, one-way ANOVA). FIG. 14C shows that co-administration of EtOH with DHM prevents EtOH-induced GABA_{A}R plasticity. Representative Western blot shows cell-surface expression (sur) vs. total (tot) expression of GABA_{A}R α4 subunit in cultured hippocampal neurons (DIV13-14) detected 24 hr after four treatments of vehicle, EtOH, E+D and DHM. β-actin is shown as a loading control and was not detectable on cell surfaces. FIG. 14D shows the quantification of surface GABA_{A}R α4 protein (% of vehicle). Surface signal was normalized to the respective β-actin signal (vehicle=100%). EtOH induced a 1.5-fold increase in surface expression of GABA_{A}R α4 protein, while E+D prevented this increase (n=5/group, *, p<0.05 vs. vehicle, one-way ANOVA).

FIGS. 15A and 15B show the escalated EtOH consumption in the two-bottle choice paradigm is completely prevented by adding DHM. FIG. 15A shows that EtOH consumption quickly escalated in the group exposed to EtOH/water intermittent-access to 20% EtOH. Co-administration of DHM (0.05 mg/ml) with EtOH (E+D/water) counteracted this increase. The symbols are the mean EtOH intake (g/kg/24 hr)±SEM. After 4 weeks, the E/water group was separated into two sub-groups; one continuing intermittent access EtOH, while the other one was given intermittent access to E+D. Whereas the E/water group kept a high level of EtOH consumption, the E+D/water group showed a great reduction in EtOH consumption within three doses of DHM, and became similar in EtOH consumption by the fourth dose of DHM. Note that there are no significant differences of solution consumption between water/water and D/water groups (n=6-8 rats/group. *, p<0.05, E+D/water group vs. E/water group; ^{†}, p<0.05, E+D/water from 5^{th} week vs. E/water group in weeks 5, 6 and 7; two-way RM ANOVA followed by Newman-Keuls post hoc test). FIG. 15B shows the plasma [EtOH] measured at the 5^{th} week (n=2-5 rats/group, *, p<0.05 vs. EtOH group, student t-test).

### DETAILED DESCRIPTION OF THE INVENTION

The present invention is directed to methods and compositions for treating, inhibiting and/or reducing alcohol (ethanol, EtOH) intoxication, withdrawal from alcohol exposure and alcohol abuse which comprises the administration of dihydromyricetin (DHM).

DHM may be obtained from the Japanese Raisin Tree, Hovenia dulcis. Herbal remedies containing Hovenia dulcis extracts and purified DHM have been used to ameliorate liver injuries induced by alcohol and other chemicals, ameliorate the symptoms of alcohol hangovers, and relive alcohol intoxication. See Kawai K, et al. (1977) Experientia 33(11):1454; Hase K, et al. (1997) Biol Pharm Bull 20:381-385; Yoshikawa, et al. (1997) Yakugaku Sasshi 117(2):108-118; Ji Y, et al. (2001) Zhong Yao Cai 24:126-128; Ji Y, et al. (2002) Zhong Yao Cai 25:190-191; Chen S H, et al. (2006) Zhongguo Zhong Yao Za Zhi 31:1094-1096; Liu X L, et al. (2006) Zhongguo Zhong Yao Za Zhi 31:1097-1100; Fang H L, et al. (2007) Am J Chin Med 35:693-703; Hussain R A, et al. (1990) J Ethnopharmacol 28(1):103-115; Yoshikawa K, et al. (1993) Phytochemistry 34:1431-1433; Yoshikawa M, et al. (1996) Chem Pharm Bull (Tokyo) 44:1454-1464; Wang Y, et al. (1994) China Trad Herbal Drugs 25:306-307; and Kim K, et al. (2000) Korean J Med Crop Sci 8:225-233.

However, prior to the present invention, it was unknown whether DHM and/or any Hovenia dulcis extracts are capable of modulating GABA_{A}R plasticity caused by alcohol exposure. In fact, prior to the present invention, no study has examined the impact of DHM and/or any Hovenia dulcis extracts on GABA_{A}Rs. In addition, the prior art studies do not necessarily involve situations of chronic alcohol exposure such that it can be said that the prior art studies inherently teach or suggest the administration of DHM and/or a Hovenia dulcis extract to treat, inhibit and/or reverse some or all GABA_{A}R plasticity caused by alcohol exposure.

A variety of flavonoids, such as myricetin, quercitin, hovenitin, laricitrin, apigenin, etc., in addition to dihydromyricetin, are found in Hovenia dulcis and other plants, e.g. Kudzu, and extracts thereof that are used in herbal remedies for various conditions. Many of the beneficial effects of flavonoids with respect to alcohol exposure are the result of their antioxidant properties. Thus, it was unknown whether DHM or any compound or extract of Hovenia dulcis would have any effect on GABA_{A}R plasticity caused by chronic alcohol exposure or if the beneficial effects of DHM and extracts of Hovenia dulcis are merely a result of antioxidant activity. In addition, although we, the inventors, believed that some amounts of DHM might pass through the blood-brain barrier, it was unknown whether such amounts would have any impact on the GABA_{A}Rs as many flavenoids and antioxidants do not. Therefore, we conducted various experiments as described herein. As provided herein, the experiment show that:

1) DHM potently (1 mg/kg) counteracts EtOH intoxication. Therefore, the present invention provides methods for treating, inhibiting, reducing EtOH intoxication in a subject which comprises administering DHM to the subject in need thereof. In some embodiments, the DHM is administered before, during and/or after exposure to EtOH. In some embodiments, the DHM is administered with EtOH. For example, the DHM is added to a composition comprising the EtOH, e.g. a foodstuff such as a beverage, and then the composition is administered to the subject. In some embodiments, the EtOH intoxication is acute EtOH intoxication.

2) DHM ameliorates EtOH exposure/withdrawal-induced behavior changes, including a) tolerance to EtOH; b) increase in basal anxiety, and c) hypersensitivity to PTZ-induced seizures (hyperexcitability). Therefore, the present invention provides methods for treating a symptom caused by withdrawal from EtOH exposure which comprises administering DHM to the subject in need thereof. In some embodiments, the DHM is administered before, during and/or after exposure to EtOH has stopped. In some embodiments, the symptom is selected from the group consisting of tolerance to EtOH, increased basal anxiety, and hyperexcitability.

3) DHM prevents the escalation of EtOH consumption in subjects. Therefore, the present invention provides methods for inhibiting, reducing or preventing a subject from voluntarily consuming more EtOH which comprises administering DHM to the subject. In some embodiments, the DHM is administered before, during and/or after consumption of EtOH. In some embodiments, the DHM is administered with the EtOH to be consumed. For example, the DHM is added to a composition comprising the EtOH, e.g. a foodstuff such as a beverage, and then the composition is administered to the subject.

4) DHM does not cause intoxication, sedation or anesthesia. Therefore, the present invention provides methods for treating, reducing or preventing a decrease in alertness caused by exposure to EtOH in a subject which comprises administering DHM to the subject. In some embodiments, the DHM is administered before, during and/or after exposure to EtOH. In some embodiments, the DHM is administered with EtOH. For example, the DHM is added to a composition comprising the EtOH, e.g. a foodstuff such as a beverage, and then the composition is administered to the subject.

The experiments disclosed herein also show that: a) the counteracting effects of DHM are antagonized in vivo and in vitro by flumazenil, and DHM competitively inhibits [³H]flunitrazepam binding to the benzodiazepine site of GABA_{A}Rs; b) DHM antagonizes acute EtOH-induced potentiation of GABA_{A}Rs; c) DHM antagonizes EtOH-induced alterations in responsiveness of GABA_{A}Rs to acute EtOH including loss of I_{tonic} modulation and increased mIPSC sensitivity; d) DHM potentiates GABA_{A}Rs in hippocampal slices and cultured neurons, and retains efficacy in potentiating GABA_{A}Rs even after EtOH exposure/withdrawal which induces tolerance to EtOH; and e) DHM blocks EtOH exposure/withdrawal-induced increases in the amount of GABA_{A}R α4 subunits in rat hippocampus. In other words, DHM potentiates the activity of GABA_{A}Rs associated with EtOH exposure, antagonizes the actions of EtOH on the respective GABA_{A}Rs, and binds to the benzodiazepine site of the GABA_{A}Rs. As used herein, “potentiates” means causing an increase in the activity and/or effectiveness of the GABA_{A}Rs.

Surprisingly, the experiments herein also show that DHM inhibits, reduces, and even reverses the plasticity of GABA_{A}Rs caused by exposure to EtOH. As used herein, “plasticity” of a receptor means a change in the subunit composition of the receptor. With respect to the instant invention, as used herein, “GABA_{A}R plasticity” refers to the change in the subunit composition of GABA_{A}Rs. Exposure to EtOH causes GABA_{A}Rs containing α4βδ subunits to be internalized. When the α4 subunit returns to the postsynaptic membrane, the position of the 6 subunit is changed such that the delta subunit is no longer associated with the α4 subunit, thereby resulting in GABA_{A}R plasticity, i.e. an increase in the α4 subunit at the postsynaptic membrane as compared to that prior to EtOH exposure. As shown herein, DHM inhibits, reduces, reverses and/or prevents GABA_{A}R plasticity caused by exposure to EtOH. These results are surprising because, until the present invention, there are no known compounds or compositions which inhibit, reduce, reverse and/or prevent GABA_{A}R plasticity caused by EtOH exposure. The results of the experiments herein are especially surprising in view of the fact that other flavonoids, e.g. daidzin and quercetin, which are similar to DHM, do not exhibit activities that are the same or similar to DHM, i.e. potentiate GABA_{A}Rs, antagonize EtOH actions, and bind the benzodiazepine sites of GABA_{A}Rs.

Therefore, the present invention provides methods for treating, inhibiting, reducing, reversing and/or preventing GABA_{A}R plasticity caused by exposure to EtOH which comprises administering DHM to the brain tissue acting on GABA_{A}Rs. As used herein, “GABA_{A}R plasticity caused by EtOH exposure” refers to GABA_{A}R plasticity as described by Liang J, et al. (2007) J Neurosci. 27(45):12367-77; Zucca S and Valenzuela C F (2010) J Neurosci. 30(19):6776-81; and Shen et al. (2011) Mol Pharmacol. 79(3):432-42. In some of the embodiments of the present invention, the amount of DHM administered is an effective amount. As used herein, an “effective amount” of DHM is an amount that results in the desired effect as compared to a control—an amount that treats, inhibits, reduces and/or reverses GABA_{A}R plasticity caused by exposure to ethanol, or potentiates the activity of a GABA_{A} receptor, or antagonizes the activity of ethanol on a GABA_{A} receptor. For example, in effective amount of DHM which reverses some or all GABA_{A}R plasticity caused by exposure (including chronic intermittent exposure and single dose exposure) to EtOH is that which increases the amount of GABA_{A}Rs having a composition and/or activity that is substantially similar to or the same as the corresponding naïve GABA_{A}Rs.

A “therapeutically effective amount” of DHM is a quantity sufficient to, when administered to a subject, treat, inhibit, reduce and/or reverse GABA_{A}R plasticity caused by exposure to EtOH, or potentiate the activity of a GABA_{A}R, or antagonize the activity of ethanol on a GABA_{A}R in the subject such that the condition of the subject is an observable improvement as compared to the condition of the subject prior to the treatment or as compared to a control subject. Also, as used herein, a “therapeutically effective amount” of DHM is an amount which when administered to the subject treats a given clinical condition, e.g. ethanol intoxication, at least one symptom of alcohol withdrawal syndrome, alcohol use disorders, or alcohol abuse, in the subject as compared to a control. Typically, therapeutically effective amounts of DHM can be orally or intravenously administered daily at a dosage of about 0.002 to about 200 mg/kg, preferably about 0.1 to about 100 mg/kg, e.g. about 1 mg/kg of body weight.

Ordinarily, a dose of 0.01 to 10 mg/kg in divided doses one to four times a day, or in sustained release formulation will be effective in obtaining the desired pharmacological effect. It will be understood, however, that the specific dose levels for any particular subject will depend upon a variety of factors including the activity of the specific compound employed, the age, body weight, general health, sex, diet, time of administration, route of administration, and rate of excretion, drug combination and the severity of the particular disease and/or condition. Frequency of dosage may also vary depending on the particular disease and/or condition treated. It will also be appreciated that the effective dosage for treatment may increase or decrease over the course of a particular treatment. Changes in dosage may result and become apparent by standard diagnostic assays in clinical techniques known in the art. In some instances chronic administration may be required. Effective amounts and therapeutically effective amounts of DHM may be readily determined by one of ordinary skill by routine methods known in the art.

In some embodiments, an effective amount of DHM may be administered in the form of a foodstuff, such as a beverage. In some embodiments, the beverage contains alcohol which may be made from fermented grains (e.g., whiskey, bourbon, rye, vodka, gin and/or beer), fermented fruits (e.g., wine, brandy, sherry and cognac), sugar cane and/or sugar beets (e.g., rum), and/or fermented head of the agave (tequila). In some embodiments, an effective amount of DHM may be administered in the form of a chewing gum composition.

The pharmaceutical formulations of the invention comprise a divided dose or a single dose of DHM and may be prepared in a unit-dosage form and/or packaging appropriate for the desired mode of administration. The pharmaceutical formulations of the present invention may be administered for therapy by any suitable route including oral, rectal, nasal, topical (including buccal and sublingual), dermal, mucosal, vaginal and parenteral (including subcutaneous, intramuscular, intravenous and intradermal). It will be appreciated that the preferred route will vary with the condition and age of the recipient, the nature of the condition to be treated. For example, in some embodiments, a therapeutically effective amount of DHM may be administered to a subject in the form of a transdermal patch or an effervescent tablet (e.g. a tablet comprising an effective amount of DHM, a carbonate salt, such as sodium bicarbonate, and an acidic material, such as citric acid which results in effervescence when dissolved in a liquid such as water).

In some embodiments, the unit dose of DHM for a human subject is about 50-70 mg. Thus, in some embodiments, foodstuffs, transdermal patches, chewing gums, and/or effervescent tablets according to the present invention comprise about 50-70 mg per unit.

### Experiments

### Animals and Materials

The Institutional Animal Care and Use Committee approved all animal experiments. Male and female Sprague-Dawley (SD) rats (250-300 g) were housed in the vivarium under a 12 h light/dark cycle and had ad libitum access to food and water.

Dihydromyricetin (DHM, (2R,3R)-3,5,7-trihydroxy-2-(3,4,5-trihydroxyphenyl)-2,3-dihydrochromen-4-one) was purchased from ZR Chemical, Shanghai, China (CAS No. 27200-12-0 98% purified by HPLC). Flumazenil, picrotoxin and bicuculine were purchased from Sigma.

### Statistical Analysis

Data were from at least three independent preparations of neuron cultures and/or rats as indicated. Sigmaplot (Windows version 10.1) and SigmaStat (Windows version 3.5) were used for data display and statistical analysis. Data were expressed as mean±SEM. One-way or two-way repeated measures (RM) ANOVA with post hoc comparison analyses based on Dunnett or Newman-Keuls, and student t-test were used to determine significant differences between treatment groups and vehicle group.

### DHM Blocks Acute EtOH Intoxication and Prevents EtOH Withdrawal Symptoms

Metabolic studies performed in rats showed that there is no metabolic change (to food and water intake, urine volume and stool volume) induced by DHM (oral administration, 1 mg/kg) administration (data not shown).

The effect of DHM on EtOH-induced LORR in rats was examined using a standard LORR assay known in the art. See Kakihana R, et al. (1966) Science 154(756):1574-1575). Briefly, after drug injection, rats were placed in the supine position in a V-shaped support. LORR onset time was taken from the endpoint of drug injection (i.p.). LORR duration ended when the animal was able to flip over three times in 30 s. LORR assays were blindly performed. LORR durations were reported as mean (min)±SEM.

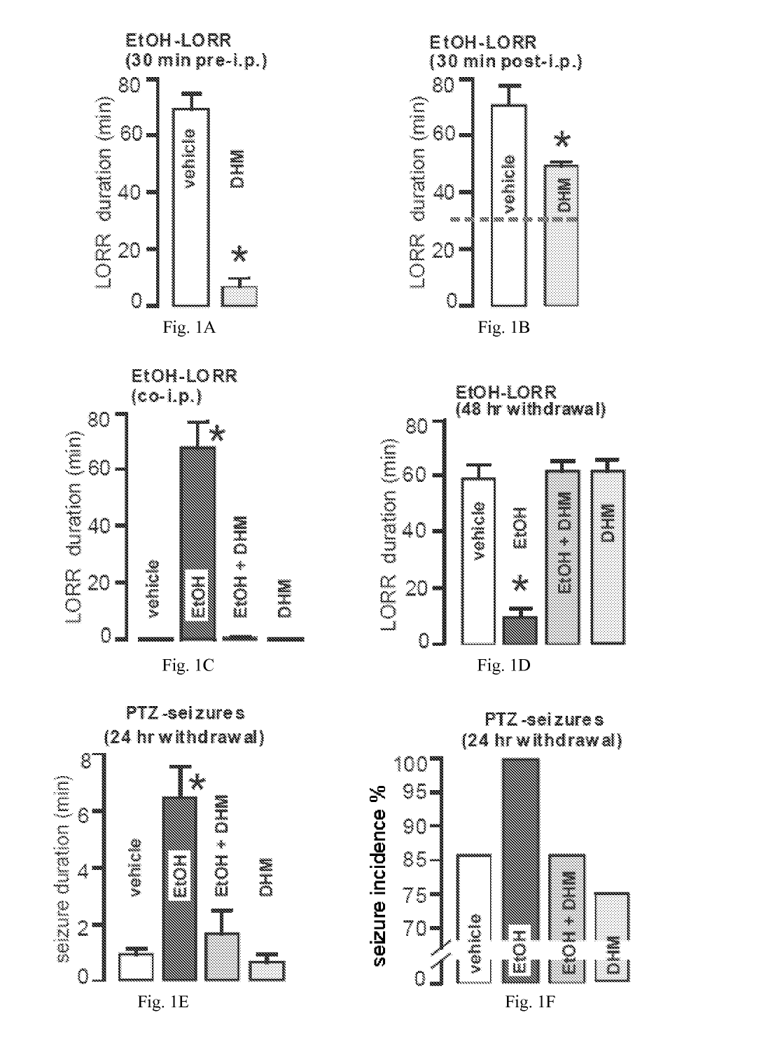

EtOH (3 g/kg, i.p.) induced 72±2 min LORR in the control group (pre-treated with saline, 20 ml/kg, i.p. 30 min prior to EtOH injection). Pre-treatment with DHM (1 mg/kg, i.p., 30 min prior to EtOH injection) the EtOH-induced LORR was reduced to 8±4 LORR (10.6±5.9% of control, FIG. 1A, p<0.05). Treatment with DHM (1 mg/kg, i.p.) 30 min after EtOH (3 mg/kg, i.p.) administration produced a reduction in LORR from 79±2 to 49±2 (FIG. 1B). In particular, starting from DHM injection (red dash line in FIG. 1B), the LORR durations were reduced from 51±2 to 21±2 min (41.2±3.8% of control, p<0.05). Co-administration of EtOH (3 mg/kg, i.p.) and DHM (1 mg/kg, i.p.) significantly reduced EtOH-induced LORR duration to 0.7±0.4 (1.2±0.6% of control, FIG. 1C, p<0.05). DHM alone did not induce LORR (FIG. 1C). These results suggest that DHM antagonize acute EtOH intoxication when administered before, during, and/or after EtOH administration.

Then the effect of DHM on single-dose EtOH-intoxication and withdrawal was examined. Rats were i.p. injection with saline (20 ml/kg, vehicle), EtOH (3 g/kg), EtOH+DHM (30 min after EtOH, 1 mg/kg), or DHM (1 mg/kg) alone. After a 48 hr withdrawal period, EtOH-induced LORR assays (EtOH, 3 g/kg, i.p.) were performed. LORR duration was significantly reduced by single-dose EtOH intoxication/withdrawal, i.e. 9±3 vs. 58±5 min (vehicle). This suggests that EtOH withdrawal induces EtOH tolerance. DHM post-treatment with EtOH significantly inhibited, reduced and/or prevented a decrease in LORR duration from EtOH withdrawal (FIG. 1D, p<0.05). LORR durations (min): EtOH+DHM 61±4 and DHM 61±4, respectively. This suggests that DHM inhibits, reduces and/or prevents EtOH withdrawal induced EtOH tolerance.

Pentylenetetrazol (PTZ)-induced seizures were also measured in rats. After 24 hr withdrawal from vehicle (saline, 20 ml/kg, i.p.), EtOH (3 g/kg, i.p.), DHM+EtOH (1 mg/kg+3 g/kg, i.p.) or DHM (1 mg/kg, i.p.) treatment, rats were tested with PTZ-induced seizures. PTZ dose used in this study (42 mg/kg in saline) was determined as the dose that induced seizures in 75% naïve rats. Briefly, after i.p. injection of PTZ, the time to onset and the duration of tonic-clonic seizures was determined as described previously. The researchers who conducted the animal behavior experiments were blind to treatment groups. Animals were used once only for any determination.

EtOH withdrawal notably increased the PTZ-seizure duration from 1.7±0.8 (vehicle) to 8.1±1.2 min (FIG. 1E, p<0.05). This suggests that EtOH withdrawal increases seizure susceptibility. The co-administration of DHM and EtOH significantly abolished, reduced, and/or inhibited increases in PTZ-seizure duration (decreased to 0.9±0.2 min). DHM pre-treatment alone did not induce any significant or observable changes in seizure duration. DHM also significantly abolished, reduced, and/or inhibited increases in seizure incidence (FIG. 1F). EtOH withdrawal increased seizure incidence to 100% compared with vehicle (85%), and DHM inhibited, reduced and/or prevented such increase (85%). These results suggest that DHM ameliorates EtOH withdrawal-induced increase in seizure susceptibility and hyperexcitability.

These findings suggest that DHM effectively inhibits, reduces, and/or prevents acute EtOH intoxication, EtOH exposure/withdrawal-induced EtOH tolerance, and EtOH withdrawal-induced hyperexcitability.

### DHM Prevents Single-Dose EtOH Intoxication-Induced GABA_{A}R Plasticity

To determine whether DHM prevents EtOH intoxication-induced alterations in GABA_{A}R sensitivity to acute EtOH, the effects of DHM on EtOH-withdrawal-induced GABA_{A}R functional alterations with whole-cell patch-clamp recording from dentate gyms granule cells (DGCs) in rat hippocampal slices at 48 hr withdrawal was examined.

Transverse slices (400 μm) of dorsal hippocampus were obtained with a Vibratome (VT 100, Technical Products International, St. Louis, Mo.) and standard techniques known in the art. Slices were continuously perfused with artificial cerebrospinal fluid (ACSF). See Liang, J., et al. (2007) J Neurosci 27:12367-12377.

Whole-cell patch-clamp recordings were obtained at 34±0.5° C. from cells located in the DG layer at a holding potential of −70 mV, during perfusion with artificial cerebrospinal fluid (ACSF, 125 mM NaCl, 2.5 mM KCl, 2 mM CaCl₂, 2 mM MgCl₂, 26 mM NaHCO₃, and 10 mM D-glucose). The ACSF was continuously bubbled with 95% O₂-5% CO₂ to ensure adequate oxygenation of slices and a pH of 7.4. Patch electrodes were pulled from thin-wall borosilicate glass pipettes with resistances of 7.5-9 MΩ and were filled with pipette solution (i.e. 137 mM CsCl, 2 mM MgCl₂, 1 mM CaCl₂, 11 mM EGTA, 10 mM HEPES and 3 mM ATP, pH adjusted to 7.30 with CsOH). Signals were recorded in voltage-clamp mode with a Axopatch 700B amplifier (Molecular Devices, Sunnyvale, Calif.). Whole cell access resistances were in the range of <25 MΩ before electrical compensation by about 70%. During voltage-clamp recordings, access resistance was monitored by measuring the size of the capacitative transient in response to a 5 mV step command and the data were abandoned if changes >20% were encountered. At least 10 min was allowed for equilibration of the pipette solution with the intracellular milieu before commencing mIPSC recordings. Intracellular signal was low-pass filtered at 3 kHz and data were acquired with Digidata 1440A and software CLAMPEX 10 (Molecular Devices) at a sampling frequency of 20 kHz.

Pharmacologically-isolated GABA_{A}R-mediated mIPSCs were recorded as previously described (Liang (2007) and Shen (2011)). For GABA concentration-response curves, evoked GABA_{A}R-currents were recorded during acute applications of GABA, DHM, or diazepam onto neurons through a removable pipette tip using a Valvelink 8.02 fast-exchange perfusion system (AutoMate Scientific, USA). Data were analyzed using the Clampfit (Version 9.0, Molecular Devices) and the MiniAnalysis Program (versions 6.0.7, Synaptosoft, Decatur, Ga.).

The MiniAnalysis program (Synaptosoft, Decatur, Ga.) was used to analyze mIPSCs. I_{tonic} is the averaged baseline currents of a given recording period. I_{tonic} amplitude was calculated as the difference between the holding currents measured before and after picrotoxin (100 μM) or bicuculline (10 μM). See Wei, W., et al. (2004) J Neurosci 24, 8379-8382; Liang (2007); and Shen (2011). Briefly, the recordings were low-pass filtered off-line (Clampfit software) at 2 kHz. The mIPSCs were detected (Mini Analysis Program, version 6.0.7) with threshold criteria of 8 pA amplitude and 20 pA*ms charge transfer. The frequency of mIPSCs was determined from all automatically detected events in a given 100 s recording period. For kinetic analysis, only single event mIPSCs with a stable baseline, sharp rising phase (10 to 90% rise time), and exponential decay were chosen during visual inspection of the recording trace. Double and multiple peak mIPSCs were excluded. At least 100 individual mIPSC events were recorded under each experimental condition. The mIPSC kinetics was obtained from analysis of the averaged chosen single events aligned with half rise time in each cell. Decay time constants were obtained by fitting a double exponential to the falling phase of the averaged mIPSCs. I_{tonic} magnitudes were obtained from the averaged baseline current of a given recording period. I_{tonic} amplitude was calculated as the difference between the holding currents measured before and after the application of picrotoxin (50 μM) or bicuculline (10 μM). See Liang J et al (2007); Shen (2011); Hamann (2002); and Mangan P S, et al. (2005) Mol Pharmacol 67(3):775-788. The investigator performing the recordings and mIPSC analysis was blind to the treatment (vehicle, EtOH, E+D, or D) that the rats received.

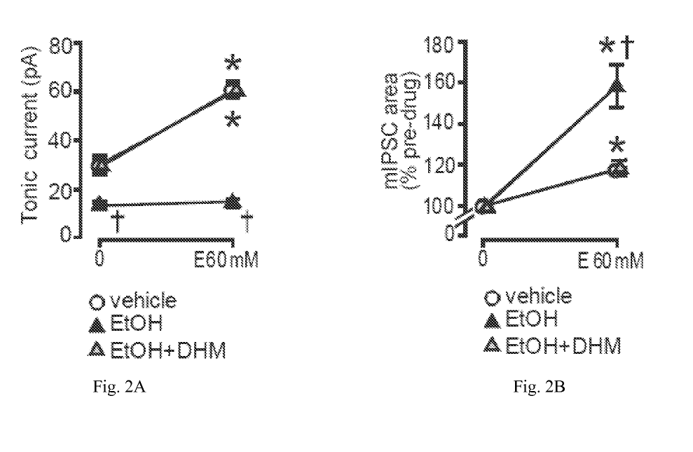

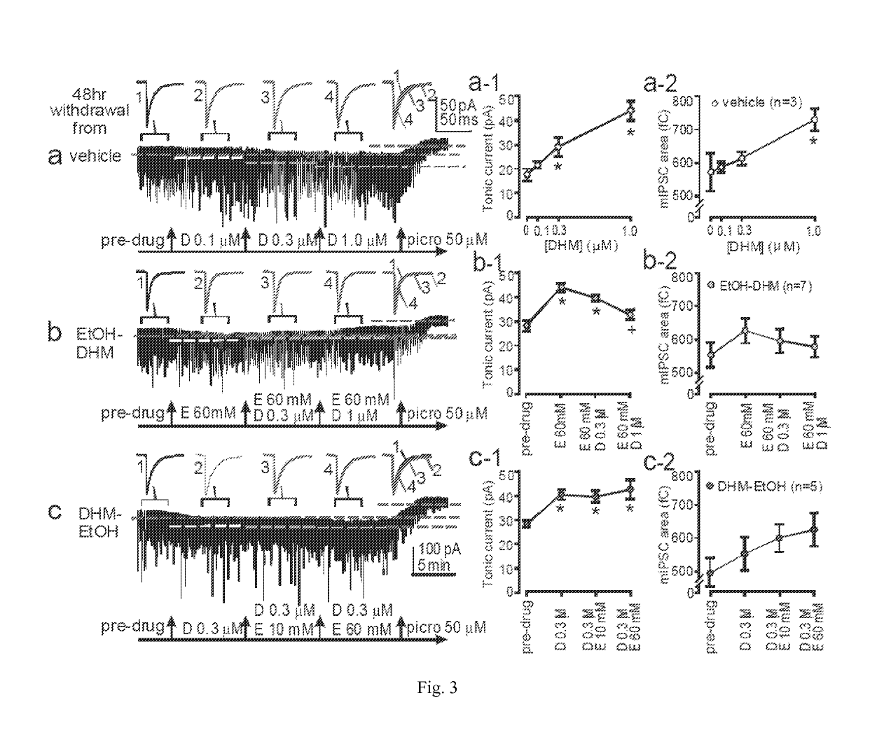

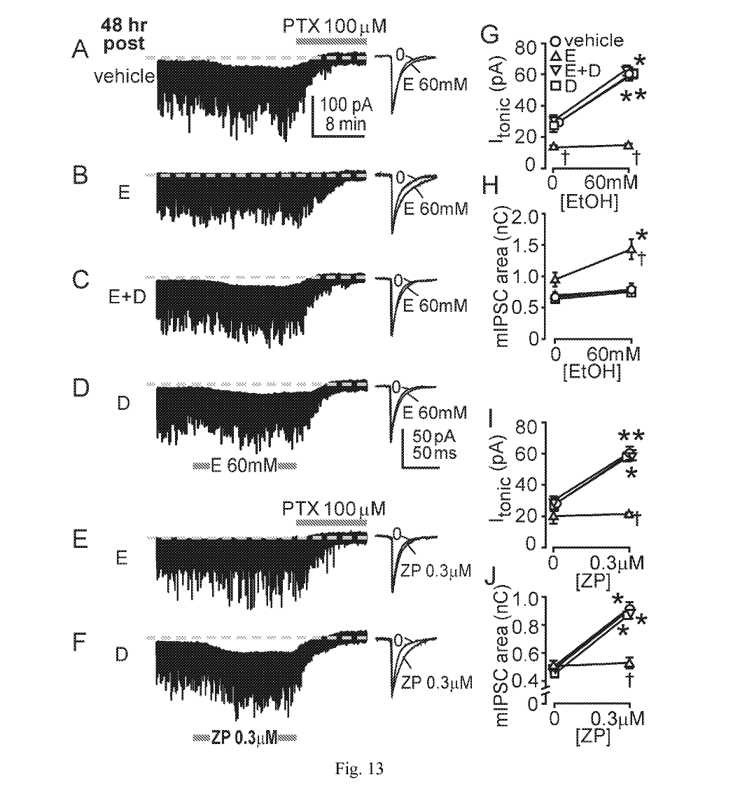

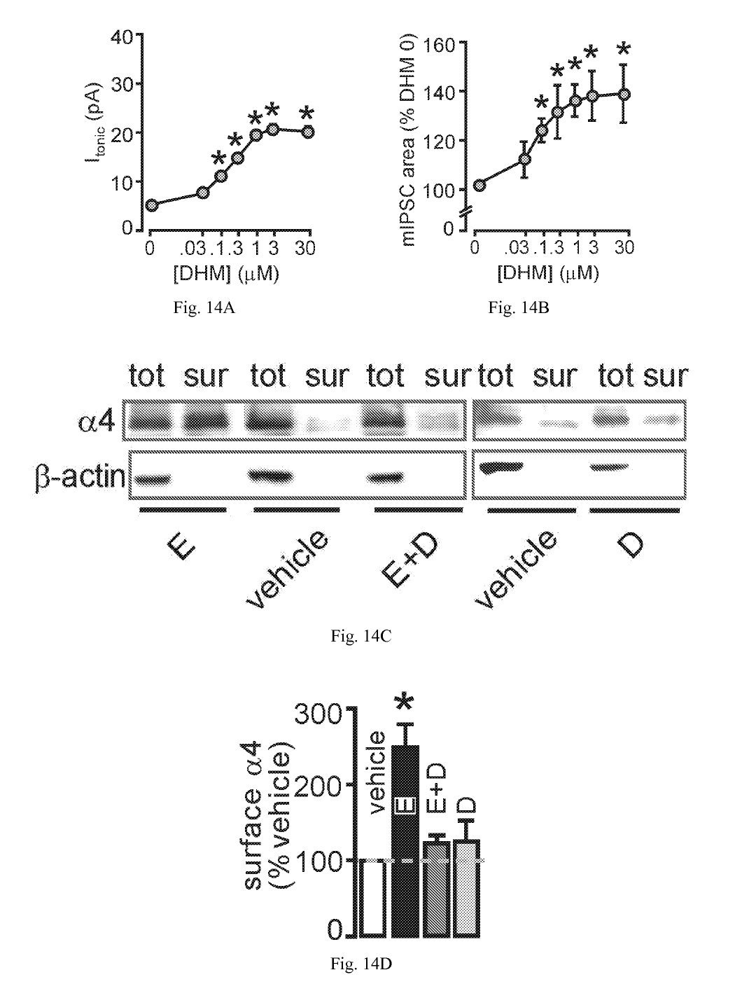

Recordings from neurons of EtOH-treated rats revealed a loss of I_{tonic} potentiation by acute EtOH (60 mM) application (FIG. 13A, FIG. 3B, FIG. 13G) (I_{tonic} from 14.0±2.1 to 14.3±2.8 pA vs. vehicle: 28.2±4.5 to 62.1±3.0 pA; FIG. 2A p<0.05), and an increase in EtOH sensitivity of mIPSCs (increased by 58.5±20.0% vs. vehicle control: 16.9±4.1% (FIG. 13H, FIG. 2B, p<0.05). By contrast, recordings from neurons of EtOH+DHM-treated rats exhibited responsiveness to acute EtOH indistinguishable from that of vehicle (FIG. 13C) (I_{tonic} increased from 27.9±3.0 to 61.3±2.3 pA, mIPSC increased by 18.0±5.9%, FIG. 13G, FIG. 13H, FIG. 2A, FIG. 2B).

Parallel Western blots from rat hippocampus were examined to determine whether EtOH induced changes in total protein of GABA_{A}R α4 subunits. Hippocampal tissues from rats were lysed in RIPA-buffer containing 1% Triton X-100, 0.1% sodium dodecyl sulfate (SDS), 50 mM Na₃PO₄, 150 mM NaCl, 2 mM EDTA, 50 mM NaF, 10 mM sodium pyrophosphate, 1 mM sodium orthovanadate, 1 mM phenylmethylsulfonyl fluoride (PMSF) and Complete protease inhibitor cocktail (Roche). The lysate was centrifuged for 15 min (14,000×g, 4° C.) and the supernatant collected for Western blot analysis. Western blots were performed using rabbit anti-GABA_{A}R α4 (aa 379-421) and mouse anti-β-actin (Sigma) followed by HRP-conjugated secondary antibodies. Bands were detected using ECL detection kit (Amersham) and analyzed by densitometric measurements using ImageQuant 5.2 (Molecular Dynamics). Bands were stripped with buffer containing 62.5 mM Tris-HCl, 100 mM β-mercaptoethanol and 2% SDS (pH 6.7) and reprobed several times. Protein concentrations were determined with BCA Protein Assay Kit (Pierce) according to the manufacturer instructions.

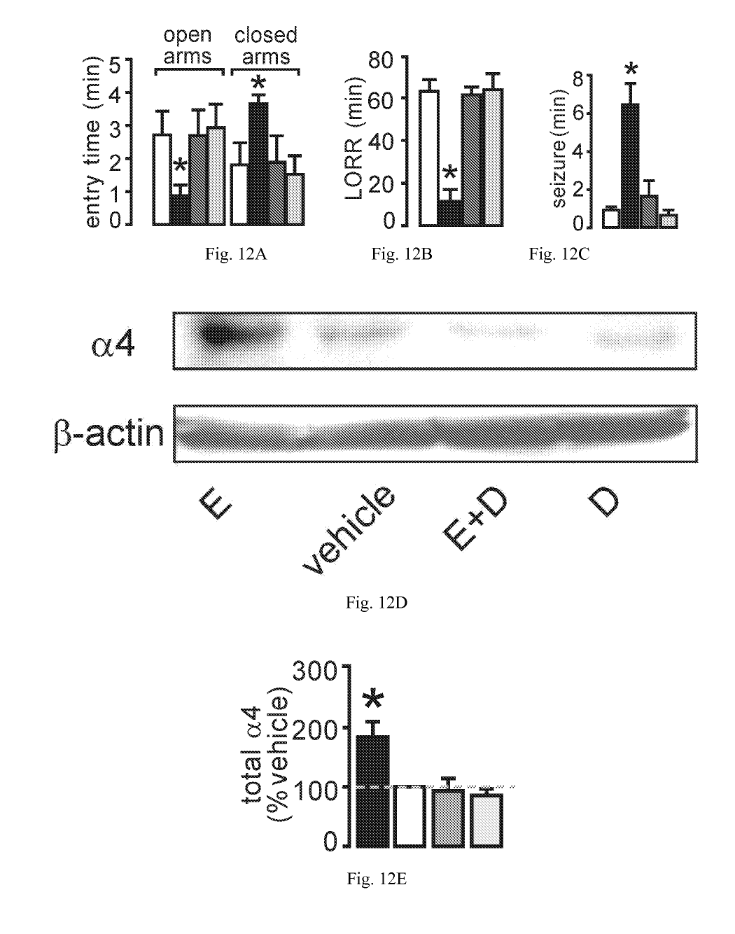

Western blots of hippocampal tissue GABA_{A}R α4 subunit after 48 hr withdrawal from rats gavaged with vehicle, EtOH, E+D or DHM are shown in FIG. 12D. EtOH exposure/withdrawal induced an increase in α4 GABA_{A}R subunit, while E+D-treatment prevented this increase. DHM alone did not produce changes in α4 GABA_{A}R subunit (FIG. 12D). FIG. 12E shows the quantification of total α4 subunit protein from the experiments of FIG. 12D. These results show that treatment of DHM with EtOH can inhibit, reduce and/or prevent all or some EtOH withdrawal-induced GABA_{A}R plasticity. Therefore, the present invention provides methods of treating, inhibiting, reducing and or preventing EtOH exposure/withdrawal-induced GABA_{A}R plasticity in a subject comprises administering to the subject DHM.

DHM Enhances GABA_{A}R-Mediated Currents, and Antagonizes their Potentiation by Acute EtOH in DGCs from Naïve Rats

To determine whether the anti-alcoholic effects of DHM result from its interaction with GABA_{A}Rs which represent a major target of alcohol actions, the effects of acute DHM on GABA_{A}R function in DGCs in hippocampal slices from naïve rats were examined as described above. Acute DHM (0.3 μM) enhanced GABA_{A}R-mediated I_{tonic} from 17.5±4.9 to 29.0±6.7 pA, prolongs mIPSC decay time and enhances mIPSC total charge transfer (area) in DGCs (area increased from 571±61 to 615±22 fC), in a concentration-dependent manner (FIG. 3, panels a, a-1, a-2).

Both EtOH and DHM potentiate GABA_{A}R-mediated currents when applied separately. However, EtOH-induced I_{tonic} potentiation was concentration-dependently decreased by DHM in the presence of EtOH (decreased from 43.8±1.8 to 32.0±2.0 pA by 1 μM DHM, FIG. 3, panels b, b-1, p<0.05). However, when EtOH was applied in the presence of DHM, there was no further potentiation in I_{tonic} (from 41.1±2.2 to 44.4±3.6 pA by 60 mM EtOH, FIG. 3, panel c, c-1).

These data indicate that DHM antagonizes EtOH intoxication-induced GABA_{A}R plasticity by interfering with EtOH-induced potentiation of GABA_{A}Rs. Therefore, the present invention provides methods of antagonizing EtOH-induced GABA_{A}R plasticity by the co-administration of DHM and EtOH. The present invention also provides methods of antagonizing EtOH-induced GABA_{A}R plasticity by administering DHM prior to exposure to EtOH. In some embodiments, the present invention provides methods for potentiating GABA_{A}R-mediated currents which comprises administering DHM.

### Co-Administration of DHM+EtOH Prevents EtOH Intoxication-Induced GABA_{A}R Plasticity in Primary Cultured Hippocampal Neurons

To determine whether DHM inhibit and/or prevent EtOH-induced GABA_{A}R plasticity in cultured neurons in vitro, the following experiment was conducted.

Hippocampal neurons from embryonic day 18 rats were prepared by papain dissociation (Worthington Biochemical, Lakewood, N.J.) and cultured in neurobasal medium and B27 supplement (Invitrogen). Cultures were kept at 37° C. in a 5% CO₂ humidified incubator as described previously. See Shen, Y., et al. (2011) Mol Pharmacol 79:432-442.

Hippocampal neurons from embryonic day 18 SD rats were prepared by papain dissociation (Worthington Biochemical, Lakewood, N.J.) and cultured in neurobasal medium (Invitrogen) and B27 supplement as previously reported. See Stowell J N and Craig A M (1999) Neuron 22(3):525-536. Briefly, embryos were removed from maternal rats anesthetized with isoflurane and euthanized by decapitation. Hippocampus were dissected and placed in Ca²⁺- and Mg²⁺-free HEPES-buffered Hank's buffered salt solution (pH 7.45). Tissues were dissociated by papain digestion followed by trituration through a Pasteur pipette and papain inhibitor treatment. Cells were pelleted and resuspended in neurobasal medium containing 2% B27 serum-free supplement, 100 U/ml penicillin, 100 μg/ml streptomycin, 0.5 mM glutamine (all from Invitrogen), and 10 μM glutamate (Sigma).

Dissociated neurons were then plated at a density of 0.3×10⁵ cells/cm² onto 12 mm round coverslips in 24-well plates (for patch-clamp recording) and/or at a density of 0.5×10⁵ cells/cm² in 6-well plates (for Western blot and biotinylation assays) coated with poly-D-lysine (Sigma, 50 μg/ml). Cultures were kept at 37° C. in a 5% CO₂ humidified incubator. Thereafter, one third to half of the medium was replaced twice a week with neurobasal culture medium containing 2% B27 supplement, and 0.5 mM glutamine.

After DIV13-14 neurons (cultured in vitro for 13-14 days), half of the medium of cultured neurons was replaced with neurobasal culture medium containing 120 mM EtOH (final EtOH concentration was 60 mM), 0.2 μM DHM plus 120 mM EtOH, or 0.2 μM DHM only (DHM control, i.e. without EtOH) for 30 min and then replaced all medium with half fresh neurobasal culture medium plus half original medium, respectively. Control neurons were treated with corresponding vehicle as same procedure as EtOH-treated neurons. The concentration of 60 mM EtOH was selected in view of prior experiments. See Liang J et al (2007). Specifically, the concentration of 60 mM EtOH used to treat cultured neurons was chosen to match blood levels measured in adult rats after intoxication with gavage of 5 g/kg, which produced about 60 mM blood peak plasma [EtOH] lasting for about 2 to 3 hr and induced significant plasticity in GABA_{A}Rs and tolerance.

DIV14 neurons (cultured in vitro for 14 days) were treated with either vehicle, EtOH, EtOH+DHM or DHM alone, followed by 24 h withdrawal. Then, immediately before electrophysiological recording, cells grown on coverslips were transferred to a perfusion chamber (Warner Instruments) and visualized with an inverted microscope (TE200, Nikon). Whole-cell patch-clamp recordings were obtained from cultured neurons under voltage-clamp mode at room temperature (22-25° C.), at a holding potential of −70 mV. Cells were perfused with an extracellular solution (137 mM NaCl, 5 mM KCl, 2 mM CaCl₂, 1 mM MgCl₂, 20 mM glucose and 10 mM HEPES (310-320 Osm, pH adjusted to 7.40 with NaOH)). Glass pipettes were filled with the same internal solution as that in slice recordings, with an input resistance of 4-7 MΩ. GABA_{A}R-mediated mIPSCs were recorded using the same pharmacological method as mentioned above. For GABA concentration-response curve, evoked GABA_{A}R-mediated currents were recorded by acute applications of GABA and/or DHM onto the cultured neurons through a removable tip that were positioned close to the soma of the neuron with a Valvelink 8.02 fast-exchange perfusion system (AutoMate Scientific, USA). Electrical signals were amplified using a Multiclamp 200 B amplifier (Molecular Devices, USA). After establishing the whole-cell configuration, at least 10 min were allowed to elapse before the application of drug to allow the membrane patch to stabilize and exchange of ions between the recording electrode and the cytosol to occur. Data were acquired with pClamp software (Version 10.0, Molecular Devices, USA), digitized at 20 kHz (Digidata 1440A, Molecular Devices), and analyzed using the Clampfit software (Version 10.0, Molecular Devices) and the Mini Analysis Program (versions 6.0.7, Synaptosoft, Decatur, Ga.) using methods known in the art. See Hamann M, et al. (2002) Neuron 33(4):625-633; Stell B M and Mody I (2002) J Neurosci 22(10): RC223; and Liang (2007).

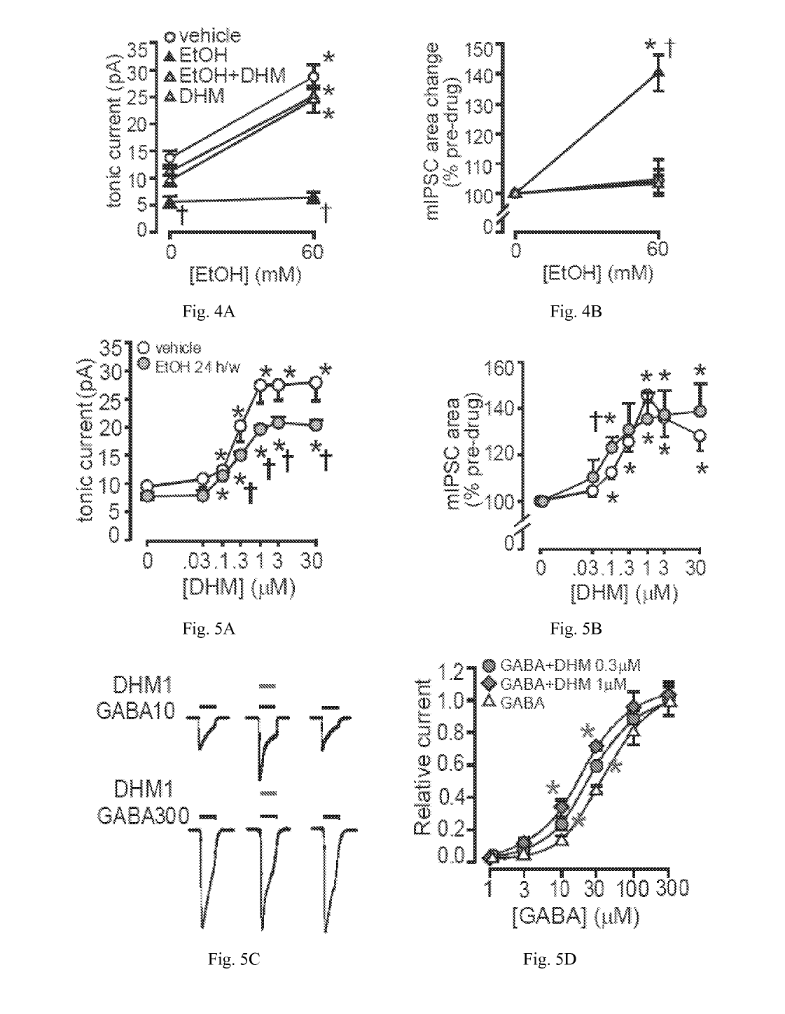

EtOH exposure/withdrawal-neurons showed dramatic decrease in I_{tonic} magnitude (from 13.8±1.4 pA in vehicle-neurons to 5.6±1.0 pA in EtOH-neurons) and in its responsiveness to acute EtOH (EtOH potentiation decreased from 109.6±15.7% in vehicle-neurons to 14.3±18.9% in EtOH-neurons, FIG. 4A, p<0.05); while EtOH exposure/withdrawal-neurons developed an increased mIPSC responsiveness to acute EtOH (EtOH potentiation increased from 3.0±10.0% in vehicle-neurons to 33.7±14.9% in EtOH-neurons, FIG. 4B, p<0.05), as previously reported. See Shen (2010). Co-administration of DHM with EtOH antagonized these effects in GABA_{A}Rs (averaged I_{tonic} magnitude was 11.2±0.6 pA, increased to 25.2±1.2 pA by EtOH, and mIPSC potentiation by EtOH was 8.3±9.4%,); DHM alone exposure/withdrawal did not alter GABA_{A}R function (FIGS. 4A and 4B).

Western blots of the cultured neurons were performed and examined as described herein. Biotinylation assays for GABA_{A}Rs of the cultured neurons were performed as described previously. See Chung W O, et al. (2000) Infect Immun 68(12):6758-6762. Briefly, the neurons in culture dishes were placed on ice and washed twice with ice cold PBS. Then the neurons were incubated for 30 min on ice with PBS containing 1 mg/ml sulfo-NHS-LC-biotin (ProteoChem). After quenching the biotin-reaction with Tris-buffered saline (TBS), neurons were lysed in 150 μl of modified RIPA-buffer (20 mM Tris-HCl (pH 7.5), 150 mM NaCl, 1 mM Na₂ EDTA, 1 mM EGTA, 1% NP-40, 1% sodium deoxycholate, 2.5 mM sodium pyrophosphate, 1 mM b-glycerophosphate, 1 mM Na₃VO₄, and 1 μg/ml leupeptin). The homogenates were centrifuged for 15 min (14,000×g, 4° C.). An aliquot (10%) of the supernatant was removed to measure β-actin. The remaining supernatant was incubated with 60 μl of 50% neutravidin agarose (Pierce Chemical Company) for 4 hr at 4° C. and washed four times with lysis buffer. Agarose-bound proteins were taken up in 20 μl of SDS sample buffer and boiled. Western blots were performed as mentioned above.

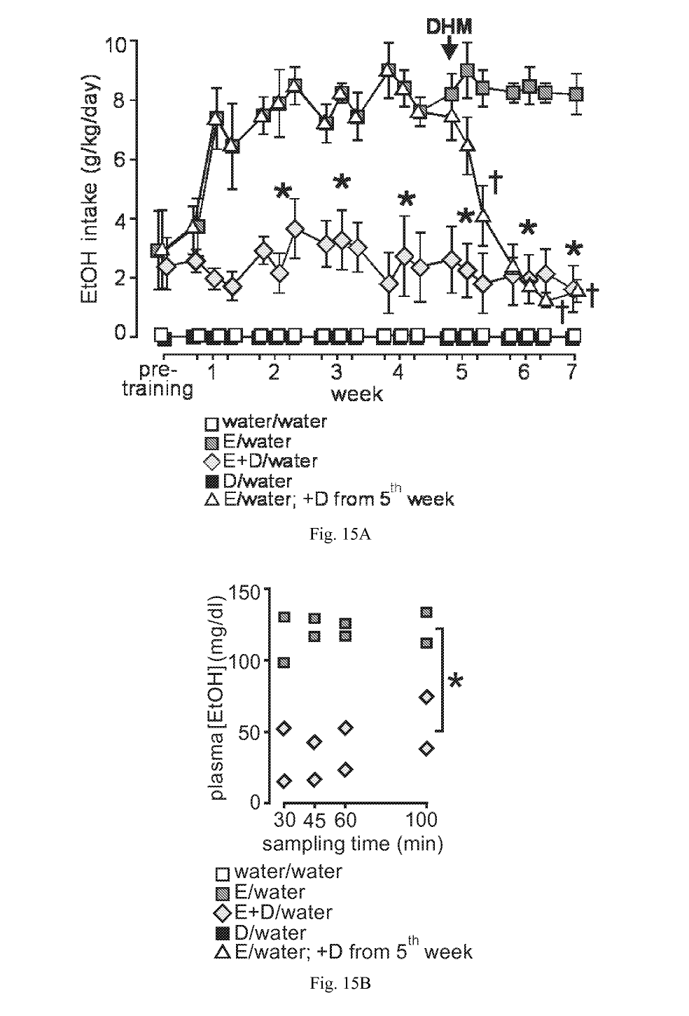

The data from the Western blots and biotinylation data showed that DHM eliminates or reverses EtOH exposure/withdrawal-induced alterations in the cell-surface GABA_{A}R α4 subunit in cultured neurons (FIG. 14C). These findings demonstrate that DHM co-administration with EtOH can inhibit, reduce and/or prevent EtOH-induced GABA_{A}R plasticity in vitro, and thereby confirm the in vivo findings in rats as disclosed herein.

### DHM Potentiates GABA_{A}R Function in Both Control Neurons and EtOH Exposure/Withdrawal-Neurons

The effect of DHM on cultured hippocampal neurons was also examined. The concentration-response curves of DHM on GABA_{A}R-mediated I_{tonic} (FIG. 5A, open circles, EC₅₀=about 0.2 μM) and mIPSCs (FIG. 5B, open circles, EC₅₀=about 0.3 μM) were established in cultured neurons. To determine whether EtOH exposure alters GABA_{A}R's responsiveness to DHM, a DHM concentration-response curve in neurons with EtOH exposure/withdrawal was also established. EtOH exposure had no effects on the concentration-response relationship of DHM with either I_{tonic} (FIG. 5A, closed circles, EC₅₀=about 0.3 μM) or mIPSCs (FIG. 5B, closed circles, EC₅₀=about 0.5 μM). These results indicate that DHM potentiates synaptic and extrasynaptic GABA_{A}Rs even in EtOH exposure/withdrawal conditions indicating DHM does not produce cross-tolerance to EtOH.

To clarify the direct effects of DHM on GABA_{A}Rs, GABA (1-300 μM) was puffed onto cultured hippocampal neurons and the concentration-response curve was established. Co-application of DHM (0.3 and 1 μM) with GABA increased the amplitude of GABA-activated currents at the same concentration of GABA, producing a left shift of the GABA concentration-response curve (FIGS. 5C and 5D). These results indicate that DHM directly potentiates GABA_{A}Rs.

### DHM Counteracts EtOH Intoxication and the DHM Effects are Antagonized by Flumazenil

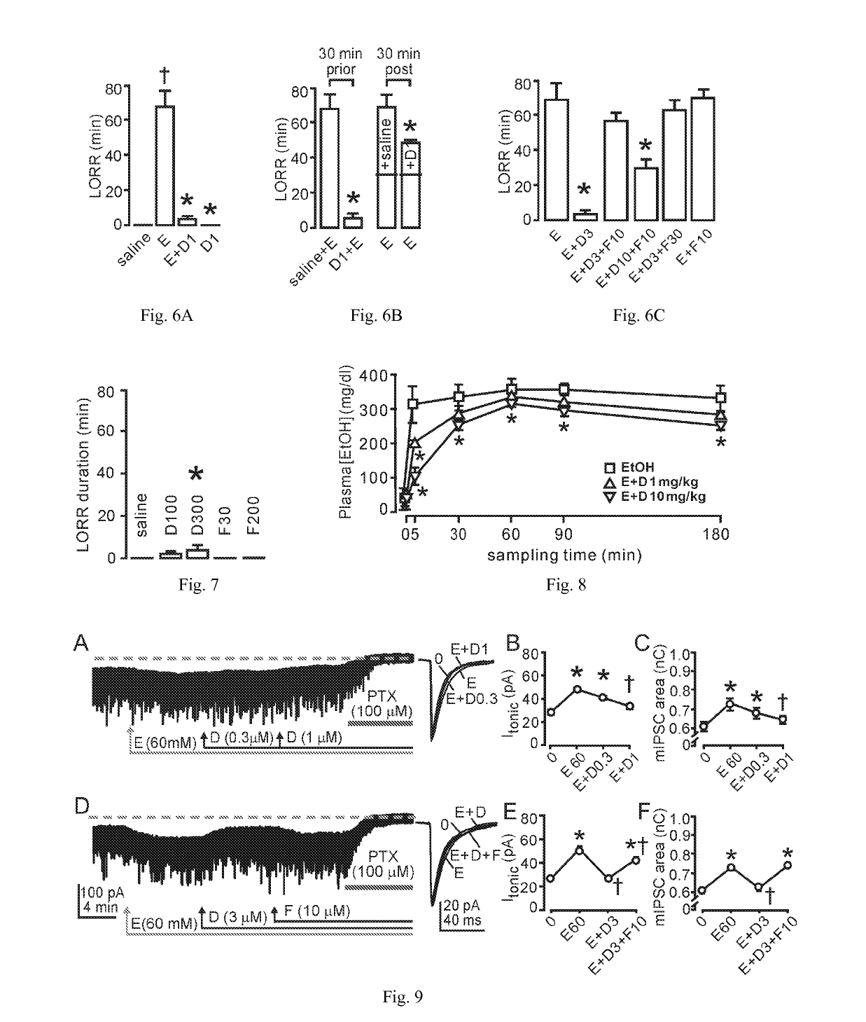

Further LORR assays were conducted as follows. Rats were divided into 4 groups and intraperitoneally (i.p.) injected with saline, EtOH (3 g/kg, E), EtOH combined with DHM (1 mg/kg, E+D1), or DHM (D1). EtOH induced 69±18 LORR. E+D1 reduced LORR to 2.7±1.4 (FIG. 6A). DHM, as saline, did not induce LORR. These results suggest that DHM counteracts acute EtOH intoxication.

Additional pre-treatment and post-treatment experiments were also conducted. 30 min prior to EtOH injection (D1+E), DHM reduced LORR to 8.2±4.1 (FIG. 6B). 30 min after injection of EtOH that induced LORR, LORR went on for an additional 42±9.1 in rats injected with saline; while injection of DHM reduced the remaining LORR to 19±1.0 (42% of E+saline, FIG. 6B). These results suggest DHM counteracts EtOH intoxication. Thus, DHM effectively ameliorates moderate to high dose EtOH intoxication even when it is administered 30 min prior or 30 min post EtOH exposure.

To examine the target of DHM's anti-EtOH effects, flumazenil, the selective benzodiazepine antagonist of modulation of GABA_{A}Rs, was tested. See Hunkeler, W., et al. (1981) Nature 290:514-516. EtOH induced 69±11.3 LORR; co-injection of DHM (3 mg/kg) and EtOH reduced LORR to 2.7±1.7 (FIG. 6C). Flumazenil (10 mg/kg) reversed the DHM reduction in LORR (56.1±4.6). Increasing DHM dose to 10 mg/kg decreased the flumazenil effect (29.3±4.8), while increasing the flumazenil dose to 30 mg/kg increased its antagonism of DHM effect (58.2±3.9). Flumazenil co-injected with EtOH did not alter LORR compared with the EtOH group (71.1±4.8, FIG. 6C). These results suggest that GABA_{A}Rs play a major role in the behavioral effects of EtOH-induced LORR in vivo. Flumazenil competitively antagonizes DHM effects on EtOH-induced LORR. In addition, the results suggest that the interactions of DHM and EtOH involve DHM action at GABA_{A}R benzodiazepine sites that may underlie DHM therapeutic effects on EtOH intoxication.

High doses of DHM (doses hundreds-fold higher than that for its antagonistic effects on EtOH intoxication) were examined. DHM (100 and 300 mg/kg) induced only 0.9±0.8 and 4.0±2.8 LORR, respectively (FIG. 7). This suggests that DHM is not merely a typical benzodiazepine. High doses of flumazenil (200 mg/kg) did not induce LORR (FIG. 7).

During the LORR assay, venous blood samples were taken at the various points from 5-180 min to measure plasma EtOH concentrations (plasma [EtOH]) from EtOH− and EtOH+DHM groups. Blood samples from the tail vein of rats at different time points (0, 5, 30, 60, 90, 180 min) after EtOH or E+D i.p. injections, or from the rats after the voluntary alcohol two-bottle choice procedure (EtOH− and EtOH+DHM group) were collected for plasma [EtOH] assays. See Liang (2007). The rat was put into a restraint tube and its tail was warm in about 40° C. The tail vein at the tip of the rail was punched with a sharp blade. Approximately 0.2 ml venous blood was dropped to a capillary blood collection tube containing lithium heparin (Ram Scientific Inc. Yonkers, N.Y.). Blood samples were centrifuged at 2500 rpm for 15 min. The supernatant was collected and stored at −80° C. until assay. The EtOH content of each blood sample was measured in duplicate along with EtOH standards using the alcohol oxidase reaction procedures (GM7 Micro-Stat; Analox Instruments, Lunenberg, Mass.).

EtOH induced onset of LORR within 5 min. Plasma [EtOH] rapidly increased for 5 min followed by a slower increase to around 60 min, then [EtOH] declined gradually. In E+D1 and E+D10 (DHM 1, 10 mg/kg) groups, the rise time of plasma [EtOH] was slowed at early time (FIG. 8). However, from 30 to 60 min, the plasma [EtOH] showed no statistically significant difference between EtOH− and E+D1-group (30 min: EtOH vs. E+D1=334.9±37.8 vs. 287.7±21.5 mg/dl, and 60 min: EtOH vs. E+D1=353.7±35.4 vs. 326.27±17.8 mg/dl), while E+D10 decreased [EtOH] significantly during that period (30 min 250.6±12.2 and 60 min 306.0±7.6 mg/dl, FIG. 8). During 30 to 60 min, the EtOH group was sleeping while E+D1- and E+D10-groups were awake. These results suggest that DHM affects EtOH pharmacokinetics, but this effect is not sufficient to account for the DHM block of EtOH-induced LORR.

### DHM Antagonizes EtOH-Induced GABA_{A}R Potentiation, and the Effect is Blocked by Flumazenil

As performed previously, whole-cell patch-clamp recordings in dentate gyms granule cells (DGCs) from hippocampal slices in vitro were conducted. Bath application of EtOH (60 mM) increased I_{tonic} from 22.0±0.7 to 46.9±1.4 pA and enhanced mIPSCs from 0.53±0.02 to 0.64±0.02 nC (FIG. 9, panels A-C). EtOH effects were concentration-dependently antagonized by DHM (0.3 and 1.0 μM).

The effect of flumazenil on the anti-EtOH actions of DHM were then tested as provided herein. DHM (3 μM) decreased EtOH-potentiated I_{tonic} from 44.8±2.3 to 21.0±0.9 pA and mIPSCs from 0.78±0.01 to 0.70±0.02 nC, while flumazenil (10 μM) reversed the DHM actions (reversed I_{tonic} to 37.3±1.6 pA, and mIPSCs to 0.78±0.01 nC, FIG. 9, panels D-F). These data suggest that DHM antagonizes EtOH-induced potentiation of both extrasynaptic and synaptic GABA_{A}Rs, and the effect is blocked by flumazenil. These data are consistent with the behavioral experiment observations (FIG. 6C) indicating that interaction of DHM and EtOH on GABA_{A}R benzodiazepine sites is a cellular mechanism underlying the therapeutic effects of DHM on EtOH intoxication.

### DHM is a Positive Modulator of GABA_{A}Rs at Benzodiazepine Sites

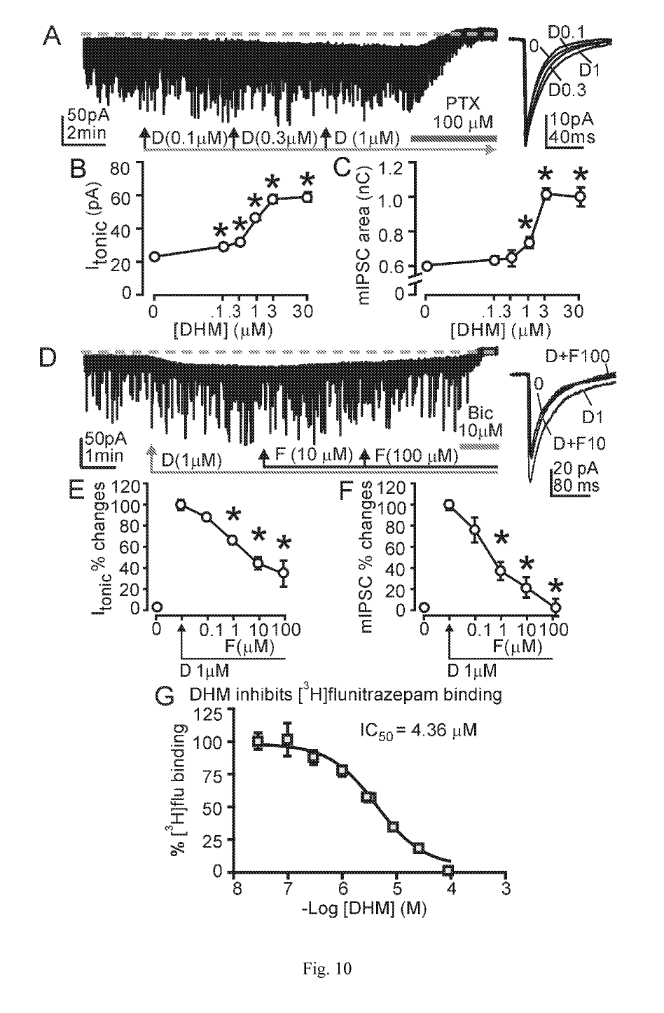

The effects of DHM (0.1 to 30 μM) on GABA_{A}R-mediated I_{tonic} and mIPSCs of DGCs in hippocampal slices. DHM (1 μM) enhanced I_{tonic} (22.5±2.5 to 44.0±4.1 pA) and increased mIPSC area (0.59±0.01 to 0.72±0.03 nC, FIG. 10, panels A-C) concentration-dependently (0.1 to 30 μM). These results indicate that DHM potentiates GABA_{A}R function in CNS neurons.

To further examine the site of DHM actions on GABA_{A}Rs, the flumazenil effects on DHM enhancing GABA_{A}R function in cultured hippocampal neurons at DIV13-14 (DIV: days in vitro) was assayed. DHM (1 μM) potentiated I_{tonic} (194.9±13.6% of control) and mIPSC area (181.8±9.2% of control, FIG. 10, panels D-F). Flumazenil inhibited the DHM enhanced GABA_{A}R-currents in a concentration-dependent manner (I_{tonic}: decreased to 143.0±3.2% and mIPSCs: to 125.7±3.9% by 10 μM flumazenil, FIG. 10, panels D-F). These observations suggest that DHM act on the same sites on GABA_{A}Rs to potentiate GABA_{A}R function as benzodiazepines.

The actions of DHM (0.03-100 μM) on the benzodiazepine sites using [³H]flunitrazepam binding in cortical membrane homogenates from naïve adult rats was examined. Standard procedures for preparation of rat cortical membranes for radioligand binding assays were conducted as previously described with modifications in speed and number of centrifugation and washes, and buffer compositions. See Li G D., et al. (2010) J Biol Chem 285:8615-8620. Naïve rat cortex was dissected from brain and homogenized in 0.32 M sucrose, 10 mM HEPES buffer (pH 7.4), and centrifuged at 650×g, 4° C. The subsequent supernatant was centrifuged at 150,000×g to collect the desired membrane-containing pellet. The pellet was washed and centrifuged two more times, first using ice-cold water and second using membrane buffer containing 50 mM KH₂PO₄, 1 mM EDTA, 2 mM benzamidine HCl, 0.5 mM DTT, 0.1 mM benzethonium HCl, 0.01% bacitracin, 0.2 PMSF (pH 7.4), and the resulting pellet was frozen. On the day of binding assay, the pellet was homogenized in assay buffer containing 50 mM KH₂PO₄, 1 mM EDTA, 200 mM KCl (pH 7.4) and centrifuged, and resuspended in fresh assay buffer to a final protein concentration of 1 mg/ml. [³H]flunitrazepam (85.2 Ci/mmol, PerkinElmer, Boston, Mass.), brain homogenate, and DHM were combined for a final assay volume of 0.5 ml, incubated on ice, and filtered by Brandel cell harvester M-24R (Brandel Co, Gaithersburg Md.). Samples were counted in a Beckman LS-3801 liquid scintillation counter. Specific binding was defined as the total amount bound (zero unlabeled ligand) minus the binding in the presence of 10 μM final concentration flurazepam (Sigma). Data was analyzed with GraphPad Prism 4.0 Software (San Diego, Calif.) to determine IC₅₀ (One-site competition equation) and Hill slope (Sigmoidal Dose-Response equation). Experiments were conducted in triplicate.

Significant inhibition of [³H]flunitrazepam binding by DHM was observed, starting at 0.3 μM in a concentration-dependent manner, with an IC₅₀ of 4.36 μM and Hill slope of −0.73 (FIG. 10, panel G). These data suggest that DHM directly inhibits [³H]flunitrazepam binding to GABA_{A}Rs, apparently competitively, indicating that DHM likely acts on GABA_{A}R benzodiazepine sites.

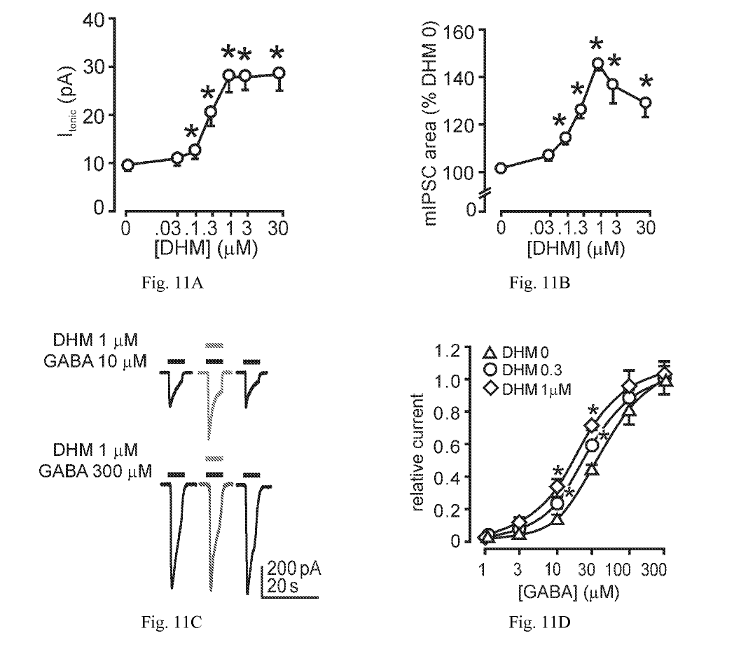

The effects of DHM on GABA_{A}R-mediated currents in cultured hippocampal neurons were also examined. DHM concentration-dependently potentiated I_{tonic} (from 9.5±1.5 to 21.0±2.3 pA by 0.3 μM DHM, EC₅₀ was about 0.20 μM) and increased mIPSCs (to 128.2±8.3% of control by 1 μM DHM, EC₅₀ was about 0.20 μM; the responses to higher than 1 μM DHM decreased slightly, FIGS. 11A and 11B).

The effects of DHM on GABA_{A}R-currents induced by focal puffs of 10 and 300 μM GABA in cultured neurons at DIV14 were examined. Co-application of DHM (0.3 and 1 M) and GABA increased the amplitude of GABA-currents and produced a left shift of the GABA concentration-response curve (FIGS. 11C and 11D). These results suggest that DHM acts on GABA_{A}Rs directly and potently potentiates synaptic and extrasynaptic GABA_{A}Rs.

### DHM Prevents EtOH Withdrawal Symptoms and Prevents EtOH Exposure/Withdrawal-Induced GABA_{A}R Plasticity in Rat Hippocampus

The effect of DHM on EtOH withdrawal symptoms in rats was examined. Rats were divided into 4 groups and gavaged with vehicle, EtOH (5 g/kg, E), EtOH combined with DHM (1 mg/kg, E+D) or DHM respectively. 48 hr after injection, rats were sub-divided into 3 groups to measure signs of EtOH withdrawal.

Anxiety and locomotion/ataxia associated with EtOH withdrawal was measured on an elevated plus-maze in EtOH-withdrawn rats (EPM, FIG. 12A). Spent time was measured in minutes. The plus-maze was constructed and the measurements were scored as described previously. See Liang et al. (2004) J Pharmacol Exp Ther 310:1234-1245. Briefly, the maze was elevated 1 m above the floor, and contained four 51 cm-long, 11.5 cm-wide arms arranged at right angles. The closed arms had opaque walls 30 cm high, extending the length of the arm. At the time of the test, each animal was placed in the center of the maze facing an open arm and allowed to explore for a 5-min session. During the session, the animal's behavior (e.g. number of arm entries and time spent in each arm per entry) was recorded on a camcorder

Subjects belonging to the vehicle group spent 2.71±0.71 in open arms and 1.80±0.67 in closed arms. Subjects belonging to the EtOH group spent significantly shorter time in the open arms (0.88±0.32) and longer time in closed arms (3.64±0.27) than vehicle group; while subjects belonging to be EtOH+DHM (E+D) group spent similar times (open: 2.68±0.77 and closed: 1.88±0.79). DHM did not affect the time rats spent in either arm (open: 2.92±0.70 and closed: 1.52±0.56). These data suggest that (1) EtOH exposure/withdrawal produces anxiety, (2) DHM combined with EtOH inhibits, reduces and/or prevents EtOH-induced anxiety, and (3) DHM does not affect anxiety levels.

Tolerance to EtOH was measured with acute EtOH-induced LORR (in minutes, FIG. 12B). EtOH induced 63.6±7.0 LORR in the vehicle group subjects as compared to 10.8±3.8 LORR in EtOH group subjects. EtOH induced 61.0±3.8 LORR in the EtOH+DHM group subjects and 65.6±8.4 LORR in DHM group subjects. These results suggest that a single exposure to EtOH produces tolerance to EtOH and DHM inhibits, reduces and/or prevents this EtOH exposure/withdrawal-induced tolerance to EtOH.

As described herein, hyperexcitability was assayed with PTZ-induced seizures duration (FIG. 12C). PTZ induced 0.9±0.2 min seizures in subjects of the vehicle group and 6.5±1.1 min seizures in subjects of the EtOH group. Seizure duration was minimized in EtOH+DHM group (1.7±0.8 min). PTZ-induced seizure in the DHM group was similar to the vehicle group (0.6±0.4 min). These results suggest that EtOH exposure/withdrawal increases seizure susceptibility (hyperexcitability) and DHM ameliorates these effects of EtOH.

The total protein content of GABA_{A}R α4 subunit in hippocampus 48 hr after the above 4 treatments was assayed. Western blots showed that EtOH exposure increased the total α4-protein level to 184.0±26.0% as compared to that of the vehicle group. There was no increase in the total α4-protein level in EtOH+DHM group (93.0±21.0% of control). DHM exposure had no effect on α4 subunit level (88.3±10.3% of control, FIGS. 12D and 12E). These data indicate that DHM inhibits, reduces and/or prevents EtOH exposure/withdrawal-induced GABA_{A}R plasticity in vivo.

Whether DHM prevents EtOH-induced GABA_{A}R plasticity in CNS neurons was assayed. Four groups of rats were gavaged with vehicle (vehicle group), EtOH (EtOH group), EtOH combined with DHM (1 mg/kg, E+D, EtOH+DHM group), or DHM (DHM group). After 48 hr withdrawal, whole-cell GABA_{A}R-mediated currents were recorded on DGCs in hippocampal slices. In the vehicle group, bath application of EtOH (60 mM) enhanced I_{tonic} from 28.8±3.1 to 62.1±3.3 pA (FIG. 13, panels A, G). EtOH enhanced mIPSC area from 0.67±0.08 to 0.78±0.10 nC (FIG. 13, panels A, H). In the EtOH group, EtOH did not increase I_{tonic} (13.0±0.95 to 13.8±1.28 pA), but greatly enhanced mIPSC area from 0.95±0.01 to 1.4±0.02 nC (FIG. 13, panels B, G, H). In the EtOH+DHM group, EtOH increased I_{tonic} from 30.0±2.8 to 60.0±2.2 pA, while mIPSC modulation was unchanged (0.70±0.03 to 0.78±0.02 nC, FIG. 13, panels C, G, H). In the DHM group, the responses of I_{tonic} and mIPSCs to EtOH were similar to those of vehicle group (FIG. 13, panels D, G, H). These results suggest that intragastric administration of EtOH combined with DHM inhibits, reduces and/or prevents both the subsequent tolerance to EtOH, and EtOH-induced GABA_{A}R plasticity.

The effect of zolpidem, an agonist of benzodiazepines, on DGCs in rats following the above 4 treatments. Zolpidem induced a potentiation of GABA_{A}R-currents in the DHM group as in the vehicle group, but did not affect GABA_{A}R-currents in EtOH group, thereby suggesting that EtOH produces cross-tolerance to zolpidem (FIG. 13, panels F, I, J). These results indicate that co-administration of EtOH and DHM inhibits, reduces and/or prevents EtOH-induced GABA_{A}R plasticity, and DHM does not produce cross-tolerance to EtOH nor to zolpidem.

The effects of DHM on cultured neurons pre-exposed to EtOH were examined. Bath application of DHM enhanced I_{tonic} and mIPSCs concentration-dependently (0.03-30 μM, FIGS. 14A and 14B). The EC₅₀ for enhancing I_{tonic} (about 0.20 μM) and mEPSCs (about 0.15 μM) were similar to those in vehicle group (FIGS. 11A and 11B). The data suggest that DHM remains effective in potentiating synaptic and extrasynaptic GABA_{A}Rs even following EtOH exposure/withdrawal that leads to tolerance to EtOH.

The surface expression of α4 subunit in cultured neurons was measured using cell-surface biotinylation followed by Western blot analysis. EtOH treated neurons showed increased α4 subunit surface expression (249.7±28.1% of control); while this increase was blocked (123.0±8.4% of control, FIGS. 14C and 14D) in neurons treated with EtOH+DHM. DHM did not alter α4 surface expression (125.0±27.3% of control, FIGS. 14C and 14D). These in vitro data indicate that co-administration of EtOH with DHM inhibits, reduces, and/or prevents GABA_{A}R plasticity that would normally result from exposure to EtOH (including a single exposure and chronic intermittent exposure to EtOH).

### DHM Reduced EtOH Consumption in a Chronic Voluntary Alcohol Intake Rat Model

The effects of DHM on alcohol consumption were examined. All fluids were presented in 100 ml graduated glass cylinders with stainless-steel drinking spouts inserted 15 min after the lights went off in the reversed light/dark cycle room. Bottles were weighed 30 min and 24 hrs after the fluids were presented. Each rat was weighed daily to monitor health and calculate the grams of ethanol intake per kilogram of body weight. Rats were divided into 4 groups and offered intermittent access to two bottle choice of water/water, 20% EtOH/water, E+D/water, or DHM/water respectively.

Rats were trained to have free two-bottle choice access to water/water, 20% (w/v) EtOH/water, EtOH+DHM (0.05 mg/ml, E+D)/water or DHM/water for two weeks. Sweetener (2 pk/L) was added to every bottle for the first week. Sweetener (1 pk/L) was added to every bottle for the second week. After training, rats were given two-bottle choice access to water/water, EtOH/water, E+D/water, or DHM/water (without sweetener for all) for three 24-hr-sessions per week (Mondays, Wednesdays and Fridays). Rats had unlimited access to two bottles of water between the EtOH-access periods. The placement of the EtOH bottle was alternated each EtOH drinking session to control for side preferences. Rats were maintained on 20% EtOH intermittent access two-bottle choice paradigm for 7 weeks (21 EtOH-access sessions). Half of EtOH group had DHM added to the EtOH bottle beginning on the fifth week (13^{th} session). The rest of the EtOH group continued EtOH-access sessions. EtOH consumption was expressed as grams of EtOH consumed per kilogram of body weight. Rats access to two bottles of water were taken as the control-group. There was no significant difference in body weight between the control and the EtOH-drinking rats at the end of the experiments.

Starting from the second week, EtOH consumption increased from 3.1±1.3 to 7.5±0.5 g/kg/day in EtOH/water-group. Co-administration of EtOH with DHM (E+D/water group) counteracted this increase in EtOH-intake (2.6±0.4 g/kg/day, FIG. 15A). After 4 weeks, EtOH/water-group was sub-divided into 2 groups: one continued with EtOH/water, while the other one was offered E+D/water. The E/water sub-group kept up the high level of EtOH-intake, while in E+D/water sub-group, EtOH consumption was greatly reduced to 1.8±1.0 g/kg/day at the end of the 5^{th} week, and 1.2±0.2 g/kg/day at the end of 6^{th} week similar to that of the group started with E+D/water (FIG. 15A). There are no significant differences in total fluid consumption between the 4 groups. These results suggest that DHM inhibits, reduces, and/or prevents excessive alcohol consumption (abuse) if taken with alcohol. DHM reduces alcohol consumption when the high voluntary EtOH consumption is already established by EtOH exposure (treats alcohol abuse).

At the end of the fourth week, plasma [EtOH] from the group of rats exposed to E+D/water was significantly lower than that from the group exposed to E/water. Plasma [EtOH] correlated well with the measured amount of EtOH consumed. Plasma [EtOH] (mg/dl) for each animal was measured following 30, 45, 60 and 100 min of voluntary 20% EtOH started at the alcohol day of the end of the 4^{th} week. Plasma [EtOH] in the two groups are significantly different (p<0.05, FIG. 15B). These data suggest that DHM inhibits, reduces and/or prevents high voluntary EtOH consumption (EtOH abuse).

Additional experiments were conducted that show that there was a reduction in EtOH (3 g/kg, i.p.)-induced LORR duration by combined-treatment with DHM (1 mg/kg, i.p., n=6 rats/group) in female rats that is similar to that of male rats. Similarly, in female rats, co-administration of DHM+EtOH similarly inhibits, reduces and/or prevents EtOH intoxication/withdrawal-induced increases in PTZ-induced seizure duration and seizure incidence.

The experiments herein show that in hippocampal neurons in cultured or slice, DHM concentration-dependently potentiated GABA_{A}R-mediated mIPSCs and tonic current. With cultured neurons, DHM caused a left shift of the GABA concentration-response relationship. These results suggest DHM potentiates both synaptic and extrasynaptic GABA_{A}Rs. However, DHM exposure/withdrawal did not induce long lasting GABA_{A}R plasticity at the cellular level. DHM does not induce intoxicated symptoms such as LORR nor causes AWS such as increase in seizures susceptibility nor induces cross-tolerance to EtOH at a dose range that is adequate to ameliorate EtOH intoxication. Therefore, DHM may be used to treat acute and chronic alcohol consumption.

In rats withdrawal from EtOH exposure, behavioral experiments in vivo show decreased seizure thresholds, anxiety, and tolerance to sedative/anesthetic drugs in a manner similar to the symptoms observed in human AWS. As disclosed herein, studies with hippocampal slices, show that there is significant I_{tonic} sensitivity to acute EtOH at 48 hr withdrawal from DHM co-administration with EtOH. Also, DHM co-administration with EtOH blocks the reduction of baseline I_{tonic} magnitude by EtOH treatment. Similar effects of DHM were found in cultured hippocampal neurons. These results from rat hippocampus show that EtOH-induced alterations in α4-containing GABA_{A}Rs are blocked by DHM. These results suggest that DHM not only antagonizes the effect of EtOH on GABA_{A}R function but also blocks α4-containing GABA_{A}Rs re-localization from extrasynapses to synapses. Therefore, DHM may be used to treat alcohol use disorders associated with GABA_{A}R plasticity resulting from exposure to EtOH.

The experiments herein show that, with naïve GABA_{A}Rs and GABA_{A}Rs that have been exposed to EtOH, DHM continues to effectively potentiate synaptic and extrasynaptic GABA_{A}Rs. Moreover, DHM blocks acute EtOH potentiation of synaptic GABA_{A}Rs in native hippocampal neurons in brain slices. These results indicate that DHM is a modulator of GABA_{A}Rs, thereby indicating that DHM may be an effective treatment for alcohol intoxication and AWS in subjects who are tolerance to other medications, such as benzodiazepines.

Comparative experiments with daidzin, quercetin, genistein, myricetin, and puerarin show that the effects of DHM on alcohol intoxication, alcohol use disorders and alcohol abuse associated with GABA_{A}Rs due to EtOH exposure may be unique. In particular, patch clamp recordings of neurons from rat hippocampal slices show that A) DHM potentiates extrasynaptic GABA_{A}R-mediated tonic current (I_{tonic}) and post-synaptic currents (mIPSCs), whereas daidzin had no effect on I_{tonic} and significantly potentiated mIPSCs. Quercetin had no effect on either I_{tonic} or mIPSCs. Thus, DHM may be used to selectively modulate extrasynaptic GABA_{A}Rs. Patch clamp recordings of neurons in rat hippocampal slices show that, in the presence of 60 mM EtOH, DHM dose-dependently blocks EtOH potentiation of GABA_{A}R-mediated I_{tonic} and mIPSCs, whereas daidzin and quercetin do not. [3H]flunitrazepam binding assay shows that DHM, daidzein and dainzin bind GABA_{A}Rs, but are significantly replaced by [3H]flunitrazepam; while genistein, myricetin, puerarin and quercetin do not bind to GABA_{A}Rs. These results indicate that DHM uniquely antagonizes alcohol potentiation of GABA_{A}Rs in CNS neurons.

To the extent necessary to understand or complete the disclosure of the present invention, all publications, patents, and patent applications mentioned herein are expressly incorporated by reference therein to the same extent as though each were individually so incorporated.

The examples and experiments disclosed herein are intended to illustrate, but not limit the invention. Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A method of treating, inhibiting, reducing and/or reversing alcohol intoxication and alcohol use disorders associated with GABA_{A}R plasticity caused by exposure to ethanol in a subject, which comprises administering dihydromyricetin to the subject's GABA_{A} receptors that will be, is, and/or have been exposed to ethanol.

2. A method of potentiating the activity of GABA_{A} receptors, which comprises administering dihydromyricetin to the GABA_{A} receptors.

3. A method of antagonizing the activity of ethanol on GABA_{A} receptors, which comprises administering dihydromyricetin to the GABA_{A} receptor before, during, and/or after exposure to the ethanol.

4. A method of treating, inhibiting and/or reducing ethanol intoxication, a symptom of alcohol withdrawal syndrome, alcohol use disorders and/or alcohol abuse in a subject, which comprises treating, inhibiting, reducing and/or reversing GABA_{A}R plasticity of the GABA_{A} receptors in the subject according to claim 1, potentiating the activity of the GABA_{A} receptors in the subject by administering dihydromyricetin to the GABA_{A} receptors, and/or antagonizing the activity of ethanol on the GABA_{A} receptors in the subject by administering dihydromyricetin to the GABA_{A} receptor before, during, and/or after exposure to the ethanol.

5. The method according to claim 4, wherein the symptom of alcohol withdrawal syndrome is selected from the group consisting of tolerance to ethanol, increased basal anxiety, and hyperexcitability.

6. The method according to claim 4, wherein the treatment reduces or inhibits a decrease in alertness, in the subject, which is caused by the exposure to ethanol.

7. The method according to claim 4, wherein the dihydromyricetin is administered in an effective amount.

8. The method according to claim 4, wherein the dihydromyricetin is administered before, during and/or after the exposure to ethanol.

9. The method according to claim 4, wherein the dihydromyricetin is administered in the form of a foodstuff, such as a beverage, which may or may not contain ethanol.

10. The method according to claim 4, wherein the dihydromyricetin is co-administered with ethanol.

11. (canceled)
